SEQUENCE LISTING (15 sequences)

SEQ ID NO: 1 - DNA, 4511 bp, synthetic construct
  misc_feature 1..4511: note = scAAV construct sequence
  source 1..4511: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagaggcgcgcccctgcaggacacacaaaaaaccaacacacagatctaatgaaaataaagatcttttattgcggccaagcttttaatgagtattcccacaagggttatcgctaatctgacatcccatattctggctggtgacctgcactttaaacagggtgccatcggcacacatgcacagcttatacctgacccaatctccggaccccagctggtctccactagaggagctgggcagtctggtagtggagaccatcacgctcccattcagaatgatgctgttttctgctttcagctccatgttgcctcccatgtgaaattcaattgtcttgcccatgatgaacacgccctcatttccccggacaatagctcgtccatccaccttgatattcaggtctgatgtagcgttactggtgattctctcagtgctggctttctggacgttcagagacttcaccccggaaggcagatgaaattcgtgtgtctcatagtcggtactgaacaggatattctgggtccggggatcaaagaactgcatgccaatgtcgctagtgattgatgttttattgttttccacagacagctttgtggttccctgctggaacacaatgggctgattgttcccggtgatcaccagattctcgtttctccgcccgccgacagtagatttgtacagtggatggatgacccccatatcggacacctgcttaaatcgcagcaggccactttcgtggaactccatagagtcacacccgtttgggccaatgcggatgacagcccaaatcaccagagtaatgatcagattaatcacggccaggataaacagcagaatgatgacgcagattgccaggtttcctttgcgccccctcaggcctgtcttatgcaggcgatcttcgtcaatagggatgtagccggctttgaaattgctgttgtgctccttattcactgatctcctctcgacggctttttctctcattgattttttcactggtccattgcttgactgctgctcggcggctgcggcggctgctgctgccatggtggtaccgggtacaattccgcagcttttagagcagaagtaacacttccgtacaggcctagaagtaaaggcaacatccactgaggagcagttctttgatttgcaccaccaccggatccgggacctgaaataaaagacaaaaagactaaacttacctgggcgcgccgctggctgctcctgagtgtctgtctgtgctgtggaggtggtggtagaatgagggcagcccctgtgcccctgggttatatagaggagcctacagggtgtgactagccaggaggggctgtccccagggaggggcccctgagagcagatgagctttcagctcgttgcccgggcaccgtgcccaccccggacccaggcgtgcagcttgcccagccccatggccttgtatgggctgccccaagggctgacttgctcactggttcctaaactaagtgctgagtctagctggcgggggacagctggcccttcgccgggaacatggaacagtaatactttgggagtcccaggcacgtataagccctggcccccaagcctgttacagcctgccctcagtcccccacagccttgttcgaagatcttcgcatgcaggggatccaccagggacagggttatttttagaggcagcaggtgttgggggggggggggcagccacatgtctgggttaattataaccaggcatctcgggtgtccccaggccttgcctccttacatgggcagcctagacccgtagtggggcatgctagacagcagggccccaaggtttgcccatgaaaggtctgttgccctcgcccctctggctccatggcctttttttagtccttgggcacattcctcctccccaaagggccgatgggcagatagaggagagacaggagcgtctcacaccacctcccctacccaggcccttacctcagttatttttaatctgaagggtctagcttagacatgcaagcttgcggccgccaattggttaaccccactccctctctgcgcgctcgctcgctcactgaggccgcccgggcaaagcccgggcgtcgggcgacctttggtcgcccggcctcagtgagcgagcgagcgcgcagagagggagtggggttaaccaattggcggccgcaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccggtaccaccatggcagcagcagccgccgcagccgccgagcagcagtcaagcaatggaccagtgaaaaaatcaatgagagaaaaagccgtcgagaggagatcagtgaataaggagcacaacagcaatttcaaagccggctacatccctattgacgaagatcgcctgcataagacaggcctgagggggcgcaaaggaaacctggcaatctgcgtcatcattctgctgtttatcctggccgtgattaatctgatcattactctggtgatttgggctgtcatccgcattggcccaaacgggtgtgactctatggagttccacgaaagtggcctgctgcgatttaagcaggtgtccgatatgggggtcatccatccactgtacaaatctactgtcggcgggcggagaaacgagaatctggtgatcaccgggaacaatcagcccattgtgttccagcagggaaccacaaagctgtctgtggaaaacaataaaacatcaatcactagcgacattggcatgcagttctttgatccccggacccagaatatcctgttcagtaccgactatgagacacacgaatttcatctgccttccggggtgaagtctctgaacgtccagaaagccagcactgagagaatcaccagtaacgctacatcagacctgaatatcaaggtggatggacgagctattgtccggggaaatgagggcgtgttcatcatgggcaagacaattgaatttcacatgggaggcaacatggagctgaaagcagaaaacagcatcattctgaatgggagcgtgatggtctccactaccagactgcccagctcctctagtggagaccagctggggtccggagattgggtcaggtataagctgtgcatgtgtgccgatggcaccctgtttaaagtgcaggtcaccagccagaatatgggatgtcagattagcgataacccttgtgggaatactcattaaaagcttggccgcaataaaagatctttattttcattagatctgtgtgttggttttttgtgtgtcctgcaggggcgcgcctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 2 - DNA, 957 bp, Homo sapiens
  source 1..957: mol_type = genomic DNA
atggcagcagcagccgccgcagccgccgagcagcagtcaagcaatggaccagtgaaaaaatcaatgagagaaaaagccgtcgagaggagatcagtgaataaggagcacaacagcaatttcaaagccggctacatccctattgacgaagatcgcctgcataagacaggcctgagggggcgcaaaggaaacctggcaatctgcgtcatcattctgctgtttatcctggccgtgattaatctgatcattactctggtgatttgggctgtcatccgcattggcccaaacgggtgtgactctatggagttccacgaaagtggcctgctgcgatttaagcaggtgtccgatatgggggtcatccatccactgtacaaatctactgtcggcgggcggagaaacgagaatctggtgatcaccgggaacaatcagcccattgtgttccagcagggaaccacaaagctgtctgtggaaaacaataaaacatcaatcactagcgacattggcatgcagttctttgatccccggacccagaatatcctgttcagtaccgactatgagacacacgaatttcatctgccttccggggtgaagtctctgaacgtccagaaagccagcactgagagaatcaccagtaacgctacatcagacctgaatatcaaggtggatggacgagctattgtccggggaaatgagggcgtgttcatcatgggcaagacaattgaatttcacatgggaggcaacatggagctgaaagcagaaaacagcatcattctgaatgggagcgtgatggtctccactaccagactgcccagctcctctagtggagaccagctggggtccggagattgggtcaggtataagctgtgcatgtgtgccgatggcaccctgtttaaagtgcaggtcaccagccagaatatgggatgtcagattagcgataacccttgtgggaatactcattaa

SEQ ID NO: 3 - protein, 318 aa, Homo sapiens
MAAAAAAAAEQQSSNGPVKKSMREKAVERRSVNKEHNSNFKAGYIPIDEDRLHKTGLRGRKGNLAICVIILLFILAVINLIITLVIWAVIRIGPNGCDSMEFHESGLLRFKQVSDMGVIHPLYKSTVGGRRNENLVITGNNQPIVFQQGTTKLSVENNKTSITSDIGMQFFDPRTQNILFSTDYETHEFHLPSGVKSLNVQKASTERITSNATSDLNIKVDGRAIVRGNEGVFIMGKTIEFHMGGNMELKAENSIILNGSVMVSTTRLPSSSSGDQLGSGDWVRYKLCMCADGTLFKVQVTSQNMGCQISDNPCGNTH

SEQ ID NO: 4 - DNA, 148 bp, synthetic construct
  misc_feature 1..148: note = 5' chimeric intron sequence
  source 1..148: mol_type = other DNA
gggtacaattccgcagcttttagagcagaagtaacacttccgtacaggcctagaagtaaaggcaacatccactgaggagcagttctttgatttgcaccaccaccggatccgggacctgaaataaaagacaaaaagactaaacttacct

SEQ ID NO: 5 - DNA, 792 bp, synthetic construct
  misc_feature 1..792: note = 5' MHCK7 promoter sequence
  source 1..792: mol_type = other DNA
gctggctgctcctgagtgtctgtctgtgctgtggaggtggtggtagaatgagggcagcccctgtgcccctgggttatatagaggagcctacagggtgtgactagccaggaggggctgtccccagggaggggcccctgagagcagatgagctttcagctcgttgcccgggcaccgtgcccaccccggacccaggcgtgcagcttgcccagccccatggccttgtatgggctgccccaagggctgacttgctcactggttcctaaactaagtgctgagtctagctggcgggggacagctggcccttcgccgggaacatggaacagtaatactttgggagtcccaggcacgtataagccctggcccccaagcctgttacagcctgccctcagtcccccacagccttgttcgaagatcttcgcatgcaggggatccaccagggacagggttatttttagaggcagcaggtgttgggggggggggggcagccacatgtctgggttaattataaccaggcatctcgggtgtccccaggccttgcctccttacatgggcagcctagacccgtagtggggcatgctagacagcagggccccaaggtttgcccatgaaaggtctgttgccctcgcccctctggctccatggcctttttttagtccttgggcacattcctcctccccaaagggccgatgggcagatagaggagagacaggagcgtctcacaccacctcccctacccaggcccttacctcagttatttttaatctgaagggtctagcttagacatgcaagctt

SEQ ID NO: 6 - DNA, 52 bp, synthetic construct
  misc_feature 1..52: note = 5' polyadenylation sequence
  source 1..52: mol_type = other DNA
acaaaaaaccaacacacagatctaatgaaaataaagatcttttattgcggcc

SEQ ID NO: 7 - DNA, 125 bp, synthetic construct
  misc_feature 1..125: note = 5' ITR
  source 1..125: mol_type = other DNA
aaccccactccctctctgcgcgctcgctcgctcactgaggccgcccgggcaaagcccgggcgtcgggcgacctttggtcgcccggcctcagtgagcgagcgagcgcgcagagagggagtggggtt

SEQ ID NO: 8 - DNA, 130 bp, synthetic construct
  misc_feature 1..130: note = 3' ITR 1
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 9 - DNA, 720 bp, synthetic construct
  misc_feature 1..720: note = tMCK
  source 1..720: mol_type = other DNA
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatcctccctggggacagcccctcctggctagtcacaccctgtaggctcctctatataacccaggggcacaggggctgcccccgggtcac

SEQ ID NO: 10 - DNA, 43 bp, synthetic construct
  misc_feature 1..43: note = Hairpin
  source 1..43: mol_type = other DNA
cgcccgggcaaagcccgggcgtcgggcgacctttggtcgcccg

SEQ ID NO: 11 - DNA, 957 bp, synthetic construct
  misc_feature 1..957: note = 5' hSGCB DNA
  source 1..957: mol_type = other DNA
ttaatgagtattcccacaagggttatcgctaatctgacatcccatattctggctggtgacctgcactttaaacagggtgccatcggcacacatgcacagcttatacctgacccaatctccggaccccagctggtctccactagaggagctgggcagtctggtagtggagaccatcacgctcccattcagaatgatgctgttttctgctttcagctccatgttgcctcccatgtgaaattcaattgtcttgcccatgatgaacacgccctcatttccccggacaatagctcgtccatccaccttgatattcaggtctgatgtagcgttactggtgattctctcagtgctggctttctggacgttcagagacttcaccccggaaggcagatgaaattcgtgtgtctcatagtcggtactgaacaggatattctgggtccggggatcaaagaactgcatgccaatgtcgctagtgattgatgttttattgttttccacagacagctttgtggttccctgctggaacacaatgggctgattgttcccggtgatcaccagattctcgtttctccgcccgccgacagtagatttgtacagtggatggatgacccccatatcggacacctgcttaaatcgcagcaggccactttcgtggaactccatagagtcacacccgtttgggccaatgcggatgacagcccaaatcaccagagtaatgatcagattaatcacggccaggataaacagcagaatgatgacgcagattgccaggtttcctttgcgccccctcaggcctgtcttatgcaggcgatcttcgtcaatagggatgtagccggctttgaaattgctgttgtgctccttattcactgatctcctctcgacggctttttctctcattgattttttcactggtccattgcttgactgctgctcggcggctgcggcggctgctgctgccat

SEQ ID NO: 12 - DNA, 148 bp, synthetic construct
  misc_feature 1..148: note = 3' chimeric intron sequence Intron
  source 1..148: mol_type = other DNA
aggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtaccc

SEQ ID NO: 13 - DNA, 792 bp, synthetic construct
  misc_feature 1..792: note = 3' MHCK7 promoter sequence MHCK7
  source 1..792: mol_type = other DNA
aagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagc

SEQ ID NO: 14 - DNA, 53 bp, synthetic construct
  misc_feature 1..53: note = 3' polyadenylation sequence PolyA
  source 1..53: mol_type = other DNA
ggccgcaataaaagatctttattttcattagatctgtgtgttggttttttgtg

SEQ ID NO: 15 - DNA, 130 bp, synthetic construct
  misc_feature 1..130: note = 3' ITR 2
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

(19) United States
(12) Patent Application Publication
(10) Pub. No.: US 2025/0288699 A1
(43) Pub. Date: Sep. 18, 2025

(54) SELF-COMPLEMENTARY ADENO-ASSOCIATED VIRUS VECTOR AND ITS USE IN TREATMENT OF MUSCULAR DYSTROPHY

(71) Applicant: RESEARCH INSTITUTE AT NATIONWIDE CHILDREN'S HOSPITAL, Columbus, OH (US)

(72) Inventors: Louise Rodino-Klapac, Columbus, OH (US); Jerry R. Mendell, Columbus, OH (US)

(73) Assignee: RESEARCH INSTITUTE AT NATIONWIDE CHILDREN'S HOSPITAL, Columbus, OH (US)

(21) Appl. No.: 19/086,011

(22) Filed: Mar. 20, 2025

Related U.S. Application Data
(63) Continuation of application No. 18/046,740, filed on Oct. 14, 2022, now Pat. No. 12,285,497.
(60) Provisional application No. 63/256,368, filed on Oct. 15, 2021.

(51) Int. Cl. A61K 48/00; A61K 38/17; A61P 21/00; C12N 15/86

(52) U.S. Cl. CPC A61K 48/0066; A61K 38/1719; A61P 21/00; C12N 15/86; C12N 2750/14143; C12N 2750/14145; C12N 2830/15

(57) ABSTRACT

Described herein are methods of treating muscular dystrophy comprising administering a self complementary recombinant AAV (rAAV) scAAVrh74.MHCK7.hSGCB vector, methods of expressing beta-sarcoglycan gene in a patient, pharmaceutical compositions comprising the rAAV, and methods of generating the rAAV.

This application is a continuation of U.S. application Ser. No. 18/046,740, filed Oct. 14, 2022, now U.S. Pat. No. 12,285,497, which claims priority benefit of U.S. Provisional Application No. 63/256,368, filed Oct. 15, 2021, all of which are incorporated by reference herein in their entirety.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: Filename:140056-0827_SL.xml; Size: 28,933 bytes; Created: May 14, 2025.

### FIELD OF THE INVENTION

Described herein are therapy vectors such as AAV vectors expressing 0-sarcoglycan and method of using these vectors to reduce and prevent fibrosis in subjects suffering from a muscular dystrophy.

### BACKGROUND

LGMDs are rare conditions and they present differently in different people with respect to age of onset, areas of muscle weakness, heart and respiratory involvement, rate of progression and severity. LGMDs can begin in childhood, adolescence, young adulthood or even later. Both genders are affected equally. LGMDs cause weakness in the shoulder and pelvic girdle, with nearby muscles in the upper legs and arms sometimes also weakening with time. Weakness of the legs often appears before that of the arms. Facial muscles are usually unaffected. As the condition progresses, people can have problems with walking and may need to use a wheelchair over time. The involvement of shoulder and arm muscles can lead to difficulty in raising arms over head and in lifting objects. In some types of LGMD, the heart and breathing muscles may be involved.

There are at least nineteen forms of LGMD, and the forms are classified by their associated genetic defects.

| Type | Pattern of Inheritance | Gene or Chromosome |
|---|---|---|
| LGMD1A | Autosomal dominant | Myotilin gene |
| LGMD1B | Autosomal dominant | Lamin A/C gene |
| LGMD1C | Autosomal dominant | Caveolin gene |
| LGMD1D | Autosomal dominant | Chromosome 7 |
| LGMD1E | Autosomal dominant | Desmin gene |
| LGMD1F | Autosomal dominant | Chromosome 7 |
| LGMD1G | Autosomal dominant | Chromosome 4 |
| LGMD2A | Autosomal recessive | Calpain-3 gene |
| LGMD2B | Autosomal recessive | Dysferlin gene |
| LGMD2C | Autosomal recessive | Gamma-sarcoglycan gene |
| LGMD2D | Autosomal recessive | Alpha-sarcoglycan gene |
| LGMD2E | Autosomal recessive | Beta-sarcoglycan gene |
| LGMD2F | Autosomal recessive | Delta-sarcoglycan gene |
| LGMD2G | Autosomal recessive | Telethonin gene |
| LGMD2H | Autosomal recessive | TRIM32 |
| LGMD2I | Autosomal recessive | FKRP gene |
| LGMD2J | Autosomal recessive | Titin gene |
| LGMD2K | Autosomal recessive | POMT1 gene |
| LGMD2L | Autosomal recessive | Fukutin gene |

Limb-girdle muscular dystrophy (LGMD) type 2E (LGMD2E) is an autosomal recessive disorder resulting from mutations in the gene encoding β-sarcoglycan (SGCB), causing loss of functional protein. LGMD2E represents a relatively common and severe form of LGMD in the United States with worldwide reports of incidence of 1/200,000-1/350,000 (Moore et al. J Neuropathol Exp Neurol 2006; 65: 995-1003). The absence of β-sarcoglycan leads to a progressive dystrophy with chronic muscle fiber loss, inflammation, fat replacement and fibrosis, all resulting in deteriorating muscle strength and function. (Araishi et al., Hum Mol Genet 1999; 8: 1589-1598, Durbee et al., Mol Cell 2000; 5: 141-151) As a complex, the sarcoglycans (α-, β, γ-, δ-), ranging in size between 35 and 50 kD, are all transmembrane proteins that provide stability to the sarcolemma offering protection from mechanical stress during muscle activity. (Araishi et al., Hum Mol Genet 1999; 8: 1589-1598) Loss of 3-sarcoglycan in LGMD2E usually results in varying degrees of concomitant loss of other sarcoglycan proteins contributing to the fragility of the muscle membrane leading to loss of myofibers.1 Although the range of clinical phenotype of LGMD2E varies, diagnosis typically occurs before age 10 and with loss of ambulation occurring by mid to late teens. Patients present with elevated serum creatine kinase (CK), proximal muscle weakness, difficulty arising from the floor and progressive loss of ambulation. Cardiac involvement occurs in as many as fifty percent of cases.

Adeno-associated virus (AAV) is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in see U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sci USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics.

An emerging form of therapy for LGMD2E is viral-mediated gene delivery to restore wild-type protein to affected muscle resulting in restoration of muscle function. Considering that a subset of patients can develop cardiomyopathy, (Fannin et al., Neuromusc Disord 2003; 13: 303-309, Sveen et al., Arch Neurol 2008; 65: 1196-1201, Melacini et al., Muscle Nerve 1999; 22: 473-479, Barresi et al., J Med Genet 2000; 37: 102-107) this would have to be considered in the long-term care of these patients. In previous reports, the Sgcb-null mouse was well characterized. Araishi et al. developed the 0-sarcoglycan-deficient mouse with accompanying loss of all of the sarcoglycans as well as sarcospan, with at least minor preservation of merosin, the dystroglycans and dystrophin, reproducing the clinical picture seen in LGMD2E. The histological changes in this animal model were also a prototype for the clinical counterpart, including the prominence of skeletal muscle fibrosis. (Gibertini et al., Cell Tissue Res 2014; 356: 427-443) Dressman et al. (Dressman et al., Hum Gene Ther 2002; 13: 1631-1646) injected the transverse abdominal muscle using rAAV2.CMV.SGCB. Expression persisted for 21 months and muscle fibers were protected from recurrent necrosis. The use of self-complementary AAV to enhance transgene expression (McCarty et al., Gene Ther 2001; 8: 1248-1254), a muscle-specific promoter to better target skeletal muscle (Wang et al., Gene Ther 2008; 15: 1489-1499; Rodino-Klapac et al., Neurology 2008; 71: 240-247) and the optimization of a human β-sarcoglycan gene (hSGCB) has also been described.

Functional improvement in patients suffering from LGMD and other muscular dystrophies require both gene restoration and reduction of fibrosis. There is a need for methods of reducing fibrosis that may be paired with gene restoration methods for more effective treatments of LGMD and other muscular dystrophies.

### SUMMARY

Described herein are gene therapy vectors, e.g. AAV, expressing the β-sarcoglycan gene and methods of delivering β-sarcoglycan to the muscle to reduce and/or prevent fibrosis; and/or to increase muscular force, and/or to treat a mammalian subject suffering from muscular dystrophy.

Provided herein are self-complementary AAV (scAAV) that express the β-sarcoglycan gene. For example, the provided scAAV comprise a polynucleotide sequence comprising i) two nucleotide sequence encoding the β-sarcoglycan protein which are self complementary, ii) two polyadenylation sequences which are self complementary and comprises a mutated inverted terminal repeat (ITR) located in center of the AAV genome sequence (expression cassette).

Also provided is a recombinant AAV (rAAV) vector comprising a polynucleotide sequence, wherein the polynucleotide sequence comprises, from 5′ to the 3′ direction, (1) a complementary sequence of a polyadenylation sequence; (2) a complementary sequence of a gene of interest; (3) a complementary sequence of an intron; (4) a complementary sequence of a promoter; (5) the 5′ ITR sequence; (6) the promoter; (7) the intron; (8) the gene of interest; and (9) the polyadenylation sequence; wherein the polynucleotide sequence is flanked by two 3′ ITR sequences, wherein the two 3′ ITR sequences are complementary. In one embodiment, the gene of interest comprises human sarcoglycan-0 (hSGCB), human sarcoglycan γ (hSGCG), human dysferlin, human ANO5, or calpain-3 (Cap 3) gene. In another embodiment, the promoter is a muscle specific control element. Examples of muscle specific control elements include human skeletal actin gene element, cardiac actin gene element, myocyte-specific enhancer binding factor MEF, muscle creatine kinase (MCK) promoter, MCK enhancer, truncated MCK (tMCK) promoter, tMCK enhancer, myosin heavy chain (MHC) promoter, MHCK7 promoter, C5-12 promoter, murine creatine kinase enhancer element, skeletal fast-twitch troponin c gene element, slow-twitch cardiac troponin c gene element, the slow-twitch troponin i gene element, hypoxia-inducible nuclear factor binding element, or steroid-inducible element or glucocorticoid response element (GRE).

Single strand AAV vectors (ssAAV), once in the nucleus, require cell-mediated synthesis of the second strand before it is ready for replication and transcription. However, the scAAV provided herein are superior to ssAAV in gene therapy because the scAAV bypasses the rate-limiting step of the cellular synthesis of the second-strand as required in a ssAAV.

Provided herein is a polynucleotide that comprises two self complementary nucleotide sequence (also referred to as expression cassettes), wherein in each nucleotide sequence comprises a MHCK7 promoter, a chimeric intron, the hSGCB cDNA sequence and a polyadenylation sequence and a single 5′ITR located in between the two nucleotide sequences. The 5′ ITR creates a hairpin when the nucleotide sequences hybridize.

For example, the disclosure provides for the polynucleotide sequence of SEQ ID NO: 1, which is also set out as a schematic in FIG. 1. The polynucleotide sequence of SEQ ID NO: 1 is a 4511-nucleotide sequence which comprises two hSGCB cDNA sequences (SEQ ID NO: 2 and/or SEQ ID NO: 11), which encode the amino acid sequence of SEQ ID NO: 3 and hybridize to each other, two chimeric intron sequences (SEQ ID NO: 4 and/or SEQ ID NO: 11) which hybridize to each other, and two MHCK7 promoters (SEQ ID NO: 5 and/or SEQ ID NO: 13) which hybridize to each other and two polyadenyation sequences (SEQ ID NO: 6 and/or SEQ ID NO: 14) which hybridize to each other. An ITR sequence located in the center of the polynucleotide sequence has the nucleotide sequence set out as SEQ ID NO: 7. Additional ITR sequences are set out as SEQ ID NOS: 8 and 15.

The disclosure provides for a polynucleotide sequence comprising a nucleotide sequence at least about 90%, at least about 95% or at least about 99% identical to the nucleotide sequence of SEQ ID NO: 1. The disclosure also provide for a polynucleotide sequence comprising the nucleotide sequence of SEQ ID NO: 1.

In addition, the disclosure provides for recombinant AAV (rAAV) comprising any of the disclosed polynucleotides. For example, the disclosure provides for an rAAV comprising a polynucleotide sequence comprising the nucleotide sequence at least about 90%, at least about 95% or at least about 99% identical to the nucleotide sequence of SEQ ID NO: 1. The disclosure also provide an rAAV comprising a polynucleotide sequence comprising the nucleotide sequence of SEQ ID NO: 1.

The disclosure also provides for rAAV comprising the polynucleotide sequence wherein the polynucleotide sequence comprises i) two self complementary nucleotide sequences, each encoding a gene of interest, wherein the two self complementary nucleotide sequences encoding the gene of interest flank a 5′ ITR sequence, ii) two self complementary polyadenylation sequences, wherein the polynucleotide sequence is flanked by two 3′ ITR sequences, wherein the two 3′ ITR sequences are complementary. For example, the gene of interest is GAD, MTM1, LPL, RPE, REP-1, CNGB3, P1ND4, XLRS, FVIII, FIX, FIX19, AAT, NF-κB, IFN-β, ARSA, NGF, hARSB, Neurturin, AADC, SUMF, SUMF1, OTC, FGF-4, ND4, ARSA, REPi, cytosine deaminase, HGF728, HGF723, hGAA, β-globin gene, Gag, MG1MA3, L523S, METRAP, GDNF, AQP1, PG9DP, HBB, ADA, TCR, CAR, Filgrastim, IL-12, GM-CSF, ICP34.5, PENK, R394, SST2, DCK. P53, HSC, human sarcoglycan-0 (hSGCB), human sarcoglycan γ (hSGCG), human dysferlin, human ANO5, calpain-3 (Cap 3) gene. For example, in the polynucleotide sequence the first nucleotide sequence is the complement sequence of the gene of interest and the second nucleotide sequence encoding the gene of interest is its sense sequence, so that the first nucleotide sequence and the second sequence nucleotide sequence are complementary to each other.

The recombinant AAV of claim 3, wherein the gene of interest is human sarcoglycan-0 (hSGCB), human sarcoglycan γ (hSGCG), human dysferlin, human ANO5, or calpain-3 (Cap 3) gene.

The disclosure also provides for rAAV comprising a polynucleotide, wherein the polynucleotide sequence comprises i) two self complementary nucleotide sequences, each encoding the human β-sarcoglycan (hSGCB) protein, such as the amino acid sequence of SEQ ID NO: 3, ii) two self complementary polyadenylation sequences. In some embodiments, the nucleotide sequence encoding the hSGCB protein is at least about 90%, at least about 95% or at least about 99% identical to the nucleotide sequence of SEQ ID NO: 2, or the nucleotide sequence encoding the hSGCB protein comprises the nucleotide sequence of SEQ ID NO: 2. For example, the polyadenylated sequence comprises the nucleotide sequence of SEQ ID NO: 6.

In another aspect, described herein a recombinant AAV vector comprising a polynucleotide sequence encoding β-sarcoglycan. In some embodiments, the polynucleotide sequence encoding β-sarcoglycan comprises a sequence e.g. at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to the nucleotide sequence set forth in SEQ ID NO: 5 or SEQ ID NO: 2 and encodes protein that retains β-sarcoglycan activity. In some embodiments, the polynucleotide sequence encoding β-sarcoglycan comprises the nucleotide sequence set forth in SEQ ID NO: 2. In some embodiments, the polynucleotide sequence encoding β-sarcoglycan consists of the nucleotide sequence set forth in SEQ ID NO: 5 or SEQ ID NO: 2.

In another aspect, a recombinant AAV vector described herein comprises a polynucleotide sequence encoding β-sarcoglycan that is at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to the amino acid sequence of SEQ ID NO: 3, and the protein retains β-sarcoglycan activity.

In another aspect, described herein is a recombinant AAV vector comprising a polynucleotide sequence encoding functional β-sarcoglycan that comprises a nucleotide sequence that hybridizes under stringent conditions to the nucleic acid sequence of SEQ ID NO: 5 or SEQ ID NO: 2, or a complement thereof.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015 M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989). More stringent conditions (such as higher temperature, lower ionic strength, higher formamide, or other denaturing agent) may also be used, however, the rate of hybridization will be affected. In instances wherein hybridization of deoxyoligonucleotides is concerned, additional exemplary stringent hybridization conditions include washing in 6×SSC 0.05% sodium pyrophosphate at 37° C. (for 14-base oligos), 48° C. (for 17-base oligos), 55° C. (for 20-base oligos), and 60° C. (for 23-base oligos).

When ranges are used herein for physical properties, such as molecular weight, concentration, or dosage, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary from, for example, between 1% and 15% of the stated number or numerical range.

Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO₄, (SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: A Practical Approach, Ch. 4, IRL Press Limited (Oxford, England). Hybridization conditions can be adjusted by one skilled in the art in order to accommodate these variables and allow DNAs of different sequence relatedness to form hybrids.

In addition, any of the provided rAAV comprise a polynucleotide wherein each of the two self complementary nucleotide sequences is operably linked to a muscle-specific control element, wherein the two muscle-specific control elements are self complementary. For example, wherein the muscle-specific control element is human skeletal actin gene element, cardiac actin gene element, myocyte-specific enhancer binding factor (MEF), muscle creatine kinase (MCK) promoter,MCK element, truncated MCK (tMCK) promoter, tMCK element, myosin heavy chain (MHC) promter, MHCK7 promoter (a hybrid version of MHC and MCK), C5-12 promoter (synthetic promoter), murine creatine kinase enhancer element, skeletal fast-twitch troponin c gene element, slow-twitch cardiac troponin c gene element, the slow-twitch troponin i gene element, hypoxia-inducible nuclear factor binding element, steroid-inducible element or glucocorticoid response element (GRE).

In some embodiments, the disclosed rAAV comprises a polynucleotide sequence wherein each of the two complementary nucleotide sequence is operably linked to the muscle-specific control element MCK (tMCK) promoter comprising the nucleotide sequence of SEQ ID NO: 9. In other embodiments, the disclosed rAAV comprises a polynucleotide sequence wherein each of the two complementary nucleotide sequence is operably linked to the muscle-specific control element MHCK7 promoter comprising the nucleotide sequence of SEQ ID NO: 13 or SEQ ID NO: 5.

In further embodiments, the disclosed rAAV comprise a polynucleotide sequence comprising two complementary chimeric introns. For example, the chimeric intron comprises the nucleotide sequence of SEQ ID NO: 12 or SEQ ID NO: 4.

In addition embodiment, the disclosed rAAV comprise three inverted terminal repeats (ITRs), wherein one ITR is flanked by the two complementary muscle specific control elements. For example, the ITRs may comprise SEQ ID NO: 7 and/or SEQ ID NO: 8 and/or SEQ ID NO: 15. In a particular example, the ITR that is flanked by the two self-complementary muscle specific control elements comprise the nucleotide sequence of SEQ ID NO: 7. In other embodiments, two ITR's comprise the nucleotide sequence of SEQ ID NO:8 and SEQ ID NO: 15, and one of the ITR's comprises the nucleotide sequence of SEQ ID NO: 7.

The AAV can be any serotype, for example AAV1, AAV2, AAV3, AAV4, AAV5, AAV6, AAV7, AAV8, AAV9, AAV-10, AAV-11, AAV-12, AAV-13 and AAVrh.74. Production of pseudotyped rAAV is disclosed in, for example, WO 01/83692.

Other types of rAAV variants, for example rAAV with capsid mutations, are also contemplated. See, for example, Marsic et al., Molecular Therapy, 22(11): 1900-1909 (2014).

The disclosure also provides for compositions comprising any of the disclosed rAAV or the disclosed polynucleotides. In some embodiments, the composition further comprises a pharmaceutically acceptable carrier, diluent and/or adjuvant. For example, the composition comprise any of the rAAV of the disclosure, a buffer agent, an ionic strength agent, and a surfactant.

In one aspect, described herein are methods of treating muscular dystrophy in a subject in need thereof comprising the step of administering to the subject any of the disclosed rAAV or any of the disclosed compositions.

In another aspect, the disclosure provides for methods of increasing muscular force and/or muscle mass in a subject suffering from muscular dystrophy comprising administering to the subject any of the disclosed rAAV or any of the disclosed compositions.

The disclosure also provides for methods of reducing contraction-induced injury in a subject suffering from muscular dystrophy comprising administering to the subject any of the disclosed rAAV or any of the disclosed compositions.

In addition, the disclosure provides for methods of treating β-sarcoglycanopathy in a subject comprising administering to the subject any of the disclosed rAAV or any of the disclosed compositions.

The disclosure also provided for methods of increasing beta-sarcoglycan positive fibers and/or decreasing CK level in a subject's muscle tissue comprising administering to the subject any of the disclosed rAAV or any of the disclosed compositions. For example, in any of the disclosed methods expression of the beta-sarcoglycan gene or the number of positive beta-sarcoglycan positive fibers is detected by measuring the beta-sarcoglycan protein level on a Western blot in muscle biopsies before and after administration of the rAAV. Alternatively, in any of the disclosed methods expression of beta-sarcoglycan gene or number of beta-sarcoglycan positive muscle fibers is detected by measuring the beta-sarcoglycan protein level by immunohistochemistry in muscle biopsies before and after administration of the rAAV.

In any of the disclosed methods, the subject is suffering from limb-girdle muscular dystrophy.

In any of the disclosed methods, the recombinant AAV or the composition is administered by intramuscular injection or intravenous injection. In other embodiments, the rAAV or the composition is administered systemically, such as administered intravenously.

In any of the disclosed methods, the recombinant AAV is administered at a dosage of 7.41×10¹³ vg/kg measured by qPCR using linear reference plasmid or its equivalent dosage of 2×10¹⁴ vg/kg measured by qPCR using supercoiled reference plasmid.

In another embodiment, in any of the disclosed methods, the recombinant AAV is administered at a dosage of 1.85×10¹³ vg/kg measured by qPCR using linear reference plasmid or its equivalent dosage of 5×10¹³ vg/kg measured by qPCR using supercoiled reference plasmid.

In one aspect, the disclosure provides for compositions for treating muscular dystrophy in a subject in need thereof, wherein the composition comprises any of the disclosed rAAV or any of the disclosed compositions.

In another aspect, the disclosure provides for compositions for increasing muscular force and/or muscle mass in a subject suffering from muscular dystrophy, wherein the composition comprises any of the disclosed rAAV or any of the disclosed compositions.

The disclosure also provides for compositions for reducing contraction-induced injury in a subject suffering from muscular dystrophy, wherein the composition comprises any of the disclosed rAAV or any of the disclosed compositions.

In addition, the disclosure provides for compositions for treating 3-sarcoglycanopathy in a subject, wherein the composition comprises any of the disclosed rAAV or any of the disclosed compositions.

The disclosure also provided for compositions for increasing beta-sarcoglycan positive fibers and/or decreasing CK level in a subject's muscle tissue, wherein the composition comprises any of the disclosed rAAV or any of the disclosed compositions.

For example, administration of the disclosed compositions results in an increase in expression of the beta-sarcoglycan gene or an increase in the number of positive beta-sarcoglycan positive fibers as detected by measuring the beta-sarcoglycan protein level on a Western blot in muscle biopsies before and after administration of the rAAV.

Alternatively, administration of the disclosed compositions results in an increase in expression of beta-sarcoglycan gene or an increase in the number of beta-sarcoglycan positive muscle fibers as detected by measuring the beta-sarcoglycan protein level by immunohistochemistry in muscle biopsies before and after administration of the rAAV.

In any of the disclosed compositions, the subject is suffering from limb-girdle muscular dystrophy.

In any of the disclosed compositions, the recombinant AAV or the composition is formulated for administration by intramuscular injection or intravenous injection. In other embodiments, the rAAV or the composition is formulated for systemic administration, such as intravenous administration.

In any of the disclosed compositions, the recombinant AAV is at a dosage of 7.41×10¹³ vg/kg measured by qPCR using linear reference plasmid or its equivalent dosage of 2×10¹⁴ vg/kg measured by qPCR using supercoiled reference plasmid.

In another embodiment, in any of the disclosed compositions, the recombinant AAV is at a dosage of 1.85×10¹³ vg/kg measured by qPCR using linear reference plasmid or its equivalent dosage of 5×10¹³ vg/kg measured by qPCR using supercoiled reference plasmid.

In one aspect, described herein are uses of any of the disclosed rAAV or any of the disclosed compositions for preparation of a medicament for treating muscular dystrophy in a subject in need thereof.

In another aspect, the disclosure provides for uses of any of the disclosed rAAV or any of the disclosed compositions for preparation of a medicament for increasing muscular force and/or muscle mass in a mammalian subject suffering from muscular dystrophy.

The disclosure also provides for use of any of the disclosed rAAV or any of the disclosed compositions for preparation of a medicament for reducing contraction-induced injury in a subject suffering from muscular dystrophy.

In addition, the disclosure provides for uses of any of the disclosed rAAV or any of the disclosed compositions for preparation of a medicament for treating 3-sarcoglycanopathy in a subject.

The disclosure also provided for use of any of the disclosed rAAV or any of the disclosed compositions for preparation of a medicament for increasing beta-sarcoglycan positive fibers and/or decreasing CK level in a subject's muscle tissue. For example, in any of the uses administration of the rAAV or the composition resulted in increased expression of the beta-sarcoglycan gene or the number of positive beta-sarcoglycan positive fibers as detected by measuring the beta-sarcoglycan protein level on a Western blot in muscle biopsies before and after administration of the rAAV. Alternatively, in any of the disclosed uses administration of the rAAV or composition results in increased expression of beta-sarcoglycan gene or number of beta-sarcoglycan positive muscle fibers as detected by measuring the beta-sarcoglycan protein level by immunohistochemistry in muscle biopsies before and after administration of the rAAV.

In any of the disclosed uses, the subject is suffering from limb-girdle muscular dystrophy.

In any of the disclosed uses, the recombinant AAV vector or the composition is formulated for administration by intramuscular injection or intravenous injection. In other embodiments, the rAAV or the composition is formulated for systemic administration, such as intravenous administration.

In any of the disclosed uses, the recombinant AAV is at a dosage of 7.41×10¹³ vg/kg measured by qPCR using linear reference plasmid or its equivalent dosage of 2×10¹⁴ vg/kg measured by qPCR using supercoiled reference plasmid.

In another embodiment, in any of the disclosed uses, the recombinant AAV is at a dosage of 1.85×10¹³ vg/kg measured by qPCR using linear reference plasmid or its equivalent dosage of 5×10¹³ vg/kg measured by qPCR using supercoiled reference plasmid.

In any of the disclosed methods, compositions or uses, the serum creatine kinase (CK) level in the subject is decreased after administration of the rAAV or the composition as compared to serum CK level before administration of the rAAV or the composition.

In another aspect, in any of the disclosed methods, compositions or uses, the level of beta-sarcoglycan gene expression in a cell of the subject is increased after administration of the rAAV or composition as compared to the level of beta-sarcoglycan gene expression before administration of the rAAV or composition; wherein the number of beta-sarcoglycan positive fibers in the muscle tissue of the subject is increased after administration of the rAAV as compared to the number of beta-sarcoglycan positive fibers before administration of the rAAV; or wherein motor function is improved in said subject as compared to the motor function of said subject before administration of the rAAV, and wherein the motor function is determined by a 100 meter timed walk test.

In one aspect, in any of the disclosed methods, compositions or uses the level of alpha-sarcoglycan gene expression is increased in a subject in need thereof after administration of the rAAV or composition as compared to the level of alpha-sarcoglycan gene expression before administration of the rAAV or composition. In another aspect, in any of the disclosed methods, composition or uses result in increased localization of alpha-sarcoglycan to a cell membrane in a subject in need thereof after administration of the rAAV or composition to the subject. In another aspect, in any of the disclosed methods, compositions or uses the level of alpha-sarcoglycan expression is increased in muscle tissue or the muscle function is improved in a subject in need thereof after administration of the rAAV as compared to the level of alpha-sarcoglycan expression or level of muscle function prior to administration the rAAV or composition.

In another aspect, the disclosure provides a method of increasing sarcoglycan expression in muscle tissue of a subject comprising administering to the subject any of the disclosed rAAV which encode hSGCB, and detecting increased expression of at least a second sarcoglycan in the cell membrane of the cell expressing said hSGCB. In some aspects, the second sarcoglycan is α-sarcoglycan (SGCA), 7-sarcoglycan (SGCG), or 6-sarcoglycan (SGCD).

In another aspect, provided is a method of generating the rAAV disclosed herein, comprising transferring a plasmid to a cell, wherein the plasmid comprises a nucleotide sequence that is at least about 90%, at least about 95%, or at least about 99% identical to SEQ ID NO: 1. In particular, the plasmid comprises a nucleotide sequence of SEQ ID NO: 1.

In any of the provided methods, compositions and uses, the level of beta-sarcoglycan gene expression in a cell of the subject is increased after administration of the rAAV or composition as compared to the level of beta-sarcoglycan gene expression before administration of the rAAV or composition; wherein the serum creatine kinase (CK) level in the subject is decreased after administration of the rAAV or the composition as compared to serum CK level before administration of the rAAV or composition; and/or wherein the number of beta-sarcoglycan positive fibers in the muscle tissue of the subject is increased after administration of the rAAV or composition as compared to the number of beta-sarcoglycan positive fibers before administration of the rAAV.

In another embodiment, in any of the provided methods, compositions and uses, motor function is improved in said subject as compared to the motor function of said subject before administration of the rAAV or composition, and wherein the motor function is determined by a 100 meter timed walk test. For example, motor function is improved by at least 5% in 1 month or thirty days post-gene transfer, at least 10% in 2 months or sixty days post-gene transfer, or at least 15% in 3 months or ninety days post gene transfer.

In some embodiments, the motor function is improved by at least 5%, 10%, 15%, 20%, 25%, 30%, 40%, 45%, or 50%.

For example, in any of the provided methods, compositions and uses, the systemic route of administration is an intravenous route. For example, the rAAV is administered using an intravenous route and the dose of the rAAV administered is about 1.85×10¹³ vg/kg or about 7.41×10¹³ vg/kg based on a linearized plasmid as the quantification standard or the dose of the rAAV administered is about 5×10¹³ vg/kg or about 2×10¹⁴ vg/kg based on a supercoiled plasmid as the quantification standard.

In some embodiments, the dose of rAAV administered using an intravenous route and the dose is about 1.0×10¹³ vg/kg to about 5×10¹⁴ based on a supercoiled plasmid as the quantitation standard or about 1.0×10¹³ vg/kg to about 1.0×10¹⁴ vg/kg based on a linearized plasmid as the quantitation standard.

In addition, the dose of the rAAV administered is about 1.5×10¹³ vg to about 2×10¹⁶ vg, or 1.5×10¹³ vg to 1×10¹⁶ vg, or about 1.5×10¹³ vg to about 2×10¹⁵ vg, or about 1.5×10¹³ vg to about 1×10¹⁵ vg. In addition, in any of the methods, compositions and uses, the dose of rAAV is administered at a concentration of about 10 mL/kg. In any of the methods, compositions or uses provided, the muscular dystrophy is limb-girdle muscular dystrophy.

In any of the methods, uses and compositions for treating muscular dystrophy provided, the subject is 4-15 years of age, has confirmed beta-sarcoglycan (SGCB) mutation in both alleles, was negative for AAVrh74 antibodies and/or had >40% or normal 100 meter walk test. In any of the methods, uses and compositions of treating muscular dystrophy provided, the subject is a pediatric subject. In some embodiments, the subject is a pediatric subject, such as a subject ranging in age from 1 to 10 years. In some embodiments, the subject is 4 to 15 years of age. The subject, in one embodiment, is an adolescent subject, such as a subject ranging in age from 10 to 19 years. In addition, the subject, in one embodiment, is a young adult subject such as a subject ranging in age from late teens or early twenties, such as the subject may range in age from 15 to 29 years of age. In some embodiments, the subject is a middle-aged adult or an elderly subject, such that the middle-aged adult may range in age from 25-55 years of age and the elderly subject may range in age over 50 years of age.

In some embodiments, the rAAV is administered by injection, infusion or implantation. For example, the rAAV is administered by infusion over approximately 1 to 2 hours. In addition, the rAAV is administered by an intravenous route through a peripheral limb vein.

In any of the provided methods, uses or compositions, the subject suffers from a genetic mutation in a gene encoding a sarcoglycan or a muscular dystrophy. In some aspects, the sarcoglycan is β-sarcoglycan (SGCB), α-sarcoglycan (SGCA), 7-sarcoglycan (SGCG), or 6-sarcoglycan (SGCD). In some aspects, the sarcoglycan is β-sarcoglycan or α-sarcoglycan.

In any of the provided methods, uses or compositions, the level of beta-sarcoglycan protein is increased by at least 25%, or at least 26%, or at least 27%, or at least 28%, or at least 29%, or at least 30%, or at least 31%, or at least 32%, or at least 33%, or at least 34%, or at least or 35% or at least 36%, or at least 37%, or at least 38%, or at least 39%, or at least 40%, or at least 41%, or at least 42%, or at least 43%, or at least 44%, or at least 45% or at least 46%, or at least 47%, or at least 48%, or at least 49%, or at least 50%, or at least 51%, or at least 52%, or at least 53%, or at least 54%, or at least 55% or at least 56%, or at least 57%, or at least 58%, or at least 59%, or at least 60%, or at least 63%, or at least 65%, or at least 70%, or at least 75%, or at least 80%, or at least 85%, or at least 90% or at least 95%, or at least 98% after administration of rAAV. For example, the level of the level of beta-sarcoglycan protein is increased by at least 33% as detected by measuring the beta-sarcoglycan protein level on a Western blot in muscle biopsied before and after administration of the rAAV, or the level of beta-sarcoglycan protein is increased by at least 38% or at least 39% as detected by measuring the beta-sarcoglycan protein level by immunohistochemistry in muscle biopsies before and after administration of the rAAV

In any of the methods, uses or compositions provided herein, the serum CK level in the subject is decreased after administration of the rAAV as compared to serum CK level before administration of the rAAV. For example, the serum level CK level in the subject is decreased by at least 50%, or at least 51%, or at least 52%, or at least 53%, or at least 54%, or at least 55% or at least 56%, or at least 57%, or at least 58%, or at least 59%, or at least 60%, or at least 63%, or at least 65%, or at least 70%, or at least 75%, or at least 80%, or at least 81%, or at least 82%, or at least 83%, or at least 84%, or at least 85%, or at least 86% or at least 87%, or at least 88%, or at least 89%, or at least 90% or at least 95%, or at least 98% by 60 to 90 days or 60 days or 90 days after administration of rAAV as compared to the serum CK level before administration of the rAAV.

In any of the methods, uses or compositions provided herein, the number of beta-sarcoglycan positive fibers in the muscle tissue of the subject is increased after administration of the rAAV as compared to the number of beta-sarcoglycan positive fibers before administration of the rAAV. For example, the number of beta-sarcoglycan positive fibers is detected by measuring the beta-sarcoglycan protein level by Western blot or immunohistochemistry on muscle biopsies before and after administration of the rAAV. For example, the number of beta-sarcoglycan positive fibers in the muscle tissue of the subject is increased by at least 25%, or at least 26%, or at least 27%, or at least 28%, or at least 29%, or at least 30%, or at least 31%, or at least 32%, or at least 33%, or at least 34%, or at least 35% or at least 36%, or at least 37%, or at least 38%, or at least 39%, or at least 40%, or at least 41%, or at least 42%, or at least 43%, or at least 44%, or at least 45% or at least 46%, or at least 47%, or at least 48%, or at least 49%, or at least 50%, or at least 51%, or at least 52%, or at least 53%, or at least 54%, or at least 55% or at least 56%, or at least 57%, or at least 58%, or at least 59%, or at least 60%, or at least 63%, or at least 65%, or at least 70%, or at least 75%, or at least 80%, or at least 85%, or at least 90% or at least 95%, or at least 98% after administration of rAAV.

In any of the methods, compositions and uses provided herein, the level of alpha-sarcoglycan in the subject is increased after administration of the rAAV as compared to the level of alpha-sarcoglycan before administration of the rAAV. The level of alpha-sarcoglycan is detected by measuring the alpha-sarcoglycan protein level by immunohistochemistry or Western blot on muscle biopsies before and after administration of the rAAV.

In any of the provided methods, uses or compositions for expressing beta-sarcoglycan gene in a cell, expression of the beta-sarcoglycan gene in the cell is detected by measuring the beta-sarcoglycan protein level on a Western blot or immunohistochemistry in muscle biopsies before and after administration of any of the rAAV or compositions disclosed herein. For example, the cell has more than one AAV viral copy number. In addition, the beta-sarcoglycan gene is measured in the subject by detecting greater than 1 rAAV vector genome copy per nucleus.

In any of these methods, uses, and compositions, the serum CK level in the subject is decreased by at least 82% by 60 days after administration of any of the disclosed rAAV or compositions as compared to the serum CK level before administration of the rAAV.

In any of these methods, uses, and compositions, the number of beta-sarcoglycan positive fibers is detected by measuring the beta-sarcoglycan protein level by Western blot or immunohistochemistry on muscle biopsies before and after administration of the rAAV. In addition, in any of the methods, uses and compositions, the number of beta-sarcoglycan positive fibers is measured by detecting greater than 1 rAAV vector genome copy per nucleus.

In any of these methods, uses and compositions the level of alpha-sarcoglycan is detected by measuring the alpha-sarcoglycan protein level by Western blot or immunohistochemistry on muscle biopsies before and after administration of the rAAV. In addition, in any of the provided methods, uses and compositions, alpha-sarcoglycan is colocalized to the membrane of a cell expressing a beta-sarcoglycan encoded by scAAVrh74.MHCK7.hSGCB.

Methods of producing a recombinant AAV vector particle comprising culturing a cell that is transferred with a plasmid described herein and recovering recombinant AAV particles from the supernatant of the transfected cells are also provided. Viral particles comprising any of the recombinant AAV vectors described herein are also contemplated.

In one embodiment, the method of generating the rAAV comprises transferring an AAV vector plasmid to a host cell. In another embodiment, the plasmid comprises a nucleotide sequence that is at least about 90%, at least about 95%, or at least about 99% identical to SEQ ID NO: 1. In another aspect, the disclosure provides a cell that comprising an AAV vector plasmid that comprises a nucleotide sequence of SEQ ID NO: 1. The cell described herein comprises may be an insect cell, e.g., a Drosophila cell (e.g., an S2 cell or Kc cell), a silkworm cell (e.g., a Bme21 cell), or a mosquito cell (e.g., a C6/36 cell); or a mammalian cell (preferably a human cell, e.g., a human primary cell or an established cell line). In one embodiment, the mammalian cell is a 293 cell, a COS cell, a HeLa cells, or a KB cell.

In another embodiment, the plasmid comprises a nucleotide sequence that is at least about 90%, at least about 95%, or at least about 99% identical to SEQ ID NO: 1. In some embodiments, the vector plasmid comprises a nucleotide sequence of any one of SEQ ID NO: 1. In some embodiments, the AAV vector plasmid is stably expressed in the host cell. The host cell stably harboring the AAV vector plasmid can be used to generate rAAV. In one embodiment, the AAV vector plasmid is a pAAV.MHCK7.hSGCB. KAN plasmid.

The method of producing recombinant AAV vector particles provided herein may further comprise a step of transferring a packaging plasmid and/or a helper virus to the host cell. For example, the methods further comprise a step wherein the packaging cell comprises a stably integrated AAV cap gene and/or wherein the packaging cell comprises a stably integrated AAV rep gene. The disclosurealso provides for a cell comprising a plasmid that comprises a nucleotide sequence that is at least about 90%, at least about 95%, or at least about 99% identical to SEQ ID NO: 1 or a plasmid that comprises a nucleotide sequence of SEQ ID NO: 1. Also provided is a cell comprising a nucleotide sequence of SEQ ID NO: 1.

Methods of reducing fibrosis in a subject in need thereof is also provided. In this regard, the method comprises administering a therapeutically effective amount of an rAAV vector described herein (or composition comprising an rAAV vector described herein) to the mammalian subject. In some embodiments, the subject suffers from muscular dystrophy. In some embodiments, administration of an rAAV vector described herein (or composition comprising an rAAV vector described herein) reduces fibrosis in skeletal muscle or in cardiac muscle of the subject.

The term “muscular dystrophy” as used herein refers to a disorder in which strength and muscle bulk gradually decline. Non-limiting examples of muscular dystrophy diseases may include Becker muscular dystrophy, tibial muscular dystrophy, Duchenne muscular dystrophy, Emery-Dreifuss muscular dystrophy, facioscapulohumeral muscular dystrophy, sarcoglycanopathies, congenital muscular dystrophy such as congenital muscular dystrophy due to partial LAMA2 deficiency, merosin-deficient congenital muscular dystrophy, type 1D congenital muscular dystrophy, Fukuyama congenital muscular dystrophy, limb-girdle type 1A muscular dystrophy, limb-girdle type 2A muscular dystrophy, limb-girdle type 2B muscular dystrophy, limb-girdle type 2C muscular dystrophy, limb-girdle type 2D muscular dystrophy, limb-girdle type 2E muscular dystrophy, limb-girdle type 2F muscular dystrophy, limb-girdle type 2G muscular dystrophy, limb-girdle type 2H muscular dystrophy, limb-girdle type 21 muscular dystrophy, limb-girdle type 21 muscular dystrophy, limb-girdle type 2J muscular dystrophy, limb-girdle type 2K muscular dystrophy, limb-girdle type IC muscular dystrophy, rigid spine muscular dystrophy with epidermolysis bullosa simplex, oculopharyngeal muscular dystrophy, Ullrich congenital muscular dystrophy, and Ullrich scleroatonic muscular dystrophy. In some embodiments, the subject is suffering from limb-girdle muscular dystrophy. In some embodiments, the subject is suffering from limb-girdle muscular dystrophy type 2E (LGMD2E).

The term “fibrosis” as used herein refers to the excessive or unregulated deposition of extracellular matrix (ECM) components and abnormal repair processes in tissues upon injury including skeletal muscle, cardiac muscle, liver, lung, kidney, and pancreas. The ECM components that are deposited include collagen, e.g. collagen 1, collagen 2 or collagen 3, and fibronectin.

In another aspect, described herein is a method of increasing muscular force and/or muscle mass in a mammalian subject comprising administering a therapeutically effective amount of an AAV vector described herein (or composition comprising an AAV vector described herein) to the mammalian subject. In one embodiment, the subject is a human.

In any of the provided formulations or compositions, the buffer agent comprises one or more of tris, tricine, Bis-tricine, HEPES, MOPS, TES, TAPS, PIPES, and CAPS. For example, the buffer agent comprises the tris with pH 8.0 at concentration of about 5 mM to about 40 mM or the buffer agent comprises the tris with pH 8.0 at about 20 mM.

In any of the provided formulations or compositions, the ionic strength agent comprises one or more of potassium chloride (KCl), potassium acetate, potassium sulfate, ammonium sulfate, ammonium chloride (NH₄Cl), ammonium acetate, magnesium chloride (MgCl₂), magnesium acetate, magnesium sulfate, manganese chloride (MnCl2), manganese acetate, manganese sulfate, sodium chloride (NaCl), sodium acetate, lithium chloride (LiCl), and lithium acetate. For example, the ionic strength agent comprises MgCl₂ at a concentration of about 0.2 mM to about 4 mM or the ionic strength agent comprises NaCl at a concentration of about 50 mM to about 500 mM, or the ionic strength agent comprises MgCl₂ at a concentration of about 0.2 mM to about 4 mM and NaCl at a concentration of about 50 mM to about 500 mM, or the ionic strength agent comprises MgCl₂ at a concentration of about 1 mM and NaCl at a concentration of about 200 mM.

In any of the provided formulations or compositions, the surfactant comprises one or more of a sulfonate, a sulfate, a phosphonate, a phosphate, a Poloxamer, and a cationic surfactant. For example, the Poloxamer comprises one or more of Poloxamer 124, Poloxamer 181, Poloxamer 184, Poloxamer 188, Poloxamer 237, Poloxamer 331, Poloxamer 338, and Poloxamer 407. The Poloxamer may be at a concentration of about 0.00001% to about 1%. An exemplary surfactant is Poloxamer 188 at a concentration of about 0.001%.

The foregoing paragraphs are not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. The invention includes, as an additional aspect, all embodiments of the invention narrower in scope in any way than the variations defined by specific paragraphs above. For example, where certain aspects of the invention that are described as a genus, it should be understood that every member of a genus is, individually, an aspect of the invention.

### BRIEF DESCRIPTION OF THE FIGURES

FIG. 1 provides a schematic of rAAVrh74.MHCK7.SGCB therapeutic β-sarcoglycan transgene cassette. Self-complementary AAV vector containing the codon-optimized human β-sarcoglycan gene (hSGCB). A muscle specific MHCK7 promoter drives expression. The cassette also contains a chimeric intron to augment processing and polyadenylation signal for stability.

FIG. 2A-2D provides an annotated nucleotide sequence of rAAVrh74.MHCK7.SGCB.

FIG. 3 demonstrates robust expression and sarcolemmal localization of SGCB at day 60 post-infusion of rAAVrh74.MHCK7.SGCB.

FIG. 4 demonstrates increased expression of SGCD and SGCG proteins at the sarcolemma at day 60 post-infusion of rAAVrh74.MHCK7.SGCB.

FIG. 5 demonstrates that reduction in creatine kinase (CK) after treatment with of rAAVrh74.MHCK7.SGCB (SRP-9003). LLN=lower limit of normal; ULN=upper limit of normal.

FIG. 6 demonstrates that of rAAVrh74.MHCK7.SGCB (SRP-9003) treatment results in sustained improvement in NSAD total score.

FIG. 7 demonstrates of rAAVrh74.MHCK7.SGCB (SRP-9003)-treated patients display an improvement in total NSAD score compared with natural history data.

### DETAILED DESCRIPTION

The practice of the present invention will employ, unless otherwise indicated, conventional methods of virology, microbiology, molecular biology and recombinant DNA techniques within the skill of the art. Such techniques are explained fully in the literature. See, e.g., Sambrook et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijssen, ed.); Fundamental Virology, 2nd Edition, vol. I & II (B. N. Fields and D. M. Knipe, eds.); Freshney Culture of Animal Cells, A Manual of Basic Technique (Wiley-Liss, Third Edition); and Ausubel et al. (1991) Current Protocols in Molecular Biology (Wiley Interscience, N.Y.).

### Definitions

As used herein, the singular forms “a,” “an,” and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. Reference to “a recombinant AAV” includes a mixture of two or more rAAV virions, and the like. All technical and scientific terms used herein have the same meaning as commonly understood to one of ordinary skill in the art to which this invention belongs unless clearly indicated otherwise.

The use of the term “or” in the claims is used to mean “and/or” unless explicitly indicated to refer to alternatives only, or the alternatives are mutually exclusive, although the disclosure supports a definition that refers to only alternatives and “and/or.”

Throughout this application, the term “about” is used to indicate that a value includes the statistical experimental error (standard deviation of error) for the device or method being employed to determine the value.

The term “vector” is meant to be any genetic element, such as a plasmid, phage, transposon, cosmid, chromosome, virus, virion, etc., which is capable of replication when associated with the proper control elements and which can transfer gene sequences between cells. In one embodiment, the vector is a viral vector.

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products. In one embodiment, the AAV vector is a vector derived from an adeno-associated virus serotype, including without limitation, AAV-1, AAV-2, AAV-3, AAV-4, AAV-5, AAV-6, AAV-7, AAV-8, AAV-9, AAV-10, AAV-11, AAV-12, AAV-13, AAV rh10, and AAVrh.74. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, as long as the sequences provide for functional rescue, replication and packaging.

The term “AAV helper functions” refer to AAV-derived coding sequences that can be expressed to provide AAV gene products that, in turn, function in trans for productive AAV replication. Thus, AAV helper functions comprise the major AAV open reading frames (ORFs), reps and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions are used herein to complement AAV functions in trans that are missing from AAV vectors.

By “recombinant virus” is meant a virus that has been genetically altered, e.g., by the addition or insertion of a heterologous nucleic acid sequence into the viral particle.

An “AAV virion” or “AAV viral particle” or “AAV vector particle” refers to a viral particle composed of at least one AAV capsid protein and an encapsidated polynucleotide AAV vector. The AAV virion, in one embodiment, comprises a heterologous polynucleotide (i.e. a polynucleotide other than a wild-type AAV genome such as a transgene to be delivered to a mammalian cell). Production of AAV viral particles, in some embodiments, includes production of AAV vector, as such a vector is contained within an AAV vector particle.

AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

For example, a wild-type (wt) AAV virus particle comprising a linear, single-stranded AAV nucleic acid genome associated with an AAV capsid protein coat. The AAV virion can be either a single-stranded (ss) AAV or self-complementary (SC) AAV. In one embodiment, a single-stranded AAV nucleic acid molecules of either complementary sense, e.g., “sense” or “antisense” strands, can be packaged into a AAV virion and both strands are equally infectious.

The term “recombinant AAV,” or “rAAV” is defined herein as an infectious, replication-defective virus composed of an AAV protein shell, encapsidating a heterologous nucleotide sequence of interest which is flanked on both sides by AAV ITRs. An rAAV, in one embodiment, is produced in a suitable host cell which has an AAV vector, AAV helper functions and accessory functions introduced therein. In this manner, the host cell is capable of encoding AAV polypeptides that are required for packaging the AAV vector (containing a recombinant nucleotide sequence of interest) into infectious recombinant virion particles for subsequent gene delivery.

The term “transfection” refers to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties, such as a nucleotide integration vector and other nucleic acid molecules, into suitable host cells.

The term “host cell” denotes, for example, microorganisms, yeast cells, insect. cells, and mammalian cells, that can be, or have been, used as recipients of an AAV helper construct, an AAV vector plasmid, an accessory function vector, or other transfer DNA. The term includes the progeny of the original cell which has been transfected. Thus, a “host cell” as used herein generally refers to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

The term “transduction” is used to refer to the administration/delivery of a polynucleotide of interest (e.g., a polynucleotide sequence encoding β-sarcoglycan) to a recipient cell either in vivo or in vitro, via a replication-deficient rAAV described resulting in expression of β-sarcoglycan by the recipient cell.

By “muscle cell” or “muscle tissue” is meant a cell or group of cells derived from muscle of any kind (for example, skeletal muscle and smooth muscle, e.g. from the digestive tract, urinary bladder, blood vessels or cardiac tissue). Such muscle cells may be differentiated or undifferentiated, such as myoblasts, myocytes, myotubes, cardiomyocytes and cardiomyoblasts.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

A “coding sequence” or a sequence which “encodes” a particular protein, is a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

A “nucleic acid” sequence refers to a DNA or RNA sequence. The nucleic acids include base analogues of DNA and RNA including, but not limited to 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

The term DNA “control sequences” refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters.” In one embodiment, the promoter is a muscle-specific promoter, which includes but is not limited to, a human skeletal actin gene element, a cardiac actin gene element, a desmin promoter, a skeletal alpha-actin (ASKA) promoter, a troponin I (TNNI2) promoter, a myocyte-specific enhancer binding factor mef binding element, a muscle creatine kinase (MCK) promoter, a truncated MCK (tMCK) promoter, a myosin heavy chain (MHC) promoter, a hybrid α-myosin heavy chain enhancer-/MCK enhancer-promoter (MHCK7) promoter, a C5-12 promoter, a murine creatine kinase enhancer element, a skeletal fast-twitch troponin c gene element, a slow-twitch cardiac troponin c gene element, a slow-twitch troponin i gene element, hypoxia-inducible nuclear factor (HIF)-response element (HRE), a steroid-inducible element, and a glucocorticoid response element (GRE). In another embodiment, the promoter is an MCK promoter, a tMCK promoter, or an MHCK7 promoter.

The term “operably linked” refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

A promoter “directs the transcription” of a coding sequence in a cell when RNA polymerase will bind the promoter sequence and transcribe the coding sequence into mRNA, which is then translated into the polypeptide encoded by the coding sequence.

“Expression cassette” or “expression construct” refers to an assembly which is capable of directing the expression of the sequence(s) or gene(s) of interest. The expression cassette includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single-stranded DNA, at least one multiple cloning site, and a “mammalian” origin of replication (e.g., a SV40 or adenovirus origin of replication).

By “isolated” when referring to a nucleotide sequence, is meant that the indicated molecule is present in the substantial absence of other biological macromolecules such as other nucleotide sequences, chromatin material, etc. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

For the purpose of describing the relative position of nucleotide sequences in a particular nucleic acid molecule throughout the instant application, such as when a particular nucleotide sequence is described as being situated “upstream,” “downstream,” “3,” or “5” relative to another sequence, it is to be understood that it is the position of the sequences in the “sense” or “coding” strand of a DNA molecule that is being referred to as is conventional in the art.

The terms “sequence identity”, “percent sequence identity”, or “percent identical” in the context of nucleic acid or amino acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. The percent identity of the sequences can be determined by techniques known in the art. For example, homology can be determined by a direct comparison of the sequence information between two polypeptide molecules by aligning the sequence information and using readily available computer programs such as ALIGN, ClustalW2 and BLAST. In one embodiment, when BLAST is used as the alignment tool, the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR.

The term “subject” refers to any member of the animal kingdom, which includes, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. In some embodiments, the subject is a human ranging in age from birth to 2 years, from 1 to 10 years, or ranging from 4 to 15 years, or ranging from 10 to 19 years, or from 20 to 40 years of age, or from 15 to 29 years of age or from 25-55 years, or ranging from 40 to 60 years, or over 50 years or over 60 years or over 65 years or over 70 years.

### AAV

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac., et al. Journal of translational medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics.

Recombinant AAV genomes of the disclosure comprise nucleic acid molecule of the disclosure and one or more AAV ITRs flanking a nucleic acid molecule. AAV DNA in the rAAV genomes may be from any AAV serotype for which a recombinant virus can be derived including, but not limited to, AAV serotypes AAVrh.74, AAV-1, AAV-2, AAV-3, AAV-4, AAV-5, AAV-6, AAV-7, AAV-8, AAV-9, AAV-10, AAV-11, AAV-12, AAV-13, AAVrh.10 and AAVrh.74. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art. To promote muscle-specific expression, AAVrh.74 can be used.

DNA plasmids of the disclosure comprise rAAV genomes of the disclosure. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAVrh.74, AAV-8, AAV-9, AAV-10, AAV-11, AAV-12, AAVrh.10, AAVrh.74 and AAV-13. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (i.e., infectious encapsidated rAAV particles) of the disclosure comprise a rAAV genome. In exemplary embodiments, the genomes of both rAAV lack AAV rep and cap DNA, that is, there is no AAV rep or cap DNA between the ITRs of the genomes. Examples of rAAV that may be constructed to comprise the nucleic acid molecules of the disclosure are set out in International Patent Application No. PCT/US2012/047999 (WO 2013/016352) incorporated by reference herein in its entirety.

In an exemplary embodiment, the recombinant AAV vector of the disclosure is produced by the triple transfection method (Xiao et al., J Virol 72, 2224-2232 (1998) using the AAV vector plasmids scAAV.MHCK7.hSCGB, pNLRep2-Caprh74 and pHelp, rAAV contains the hSGCB gene expression cassette flanked by AAV2 inverted terminal repeat sequences (ITR). It is this sequence that is encapsidated into AAVrh.74 virions. The plasmid contains the hSGCB sequence and the MHCK7 enhancer and core promoter elements of the muscle specific promoter to drive gene expression. The expression cassette also contains an SV40 intron (SD/SA) to promote high-level gene expression and the bovine growth hormone polyadenylation signal is used for efficient transcription termination.

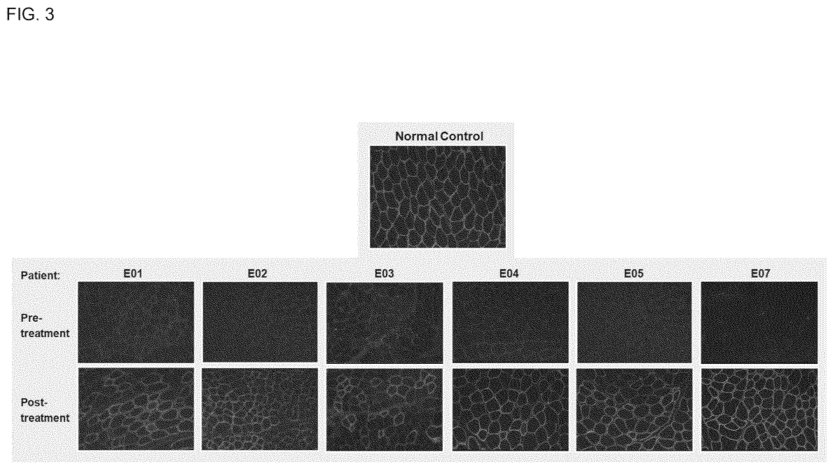

The pNLREP2-Caprh74 is an AAV helper plasmid that encodes the 4 wild-type AAV2 rep proteins and the 3 wild-type AAV VP capsid proteins from serotype rh74. A schematic map of the pNLREP2-Caprh74 plasmid is shown in FIG. 3.

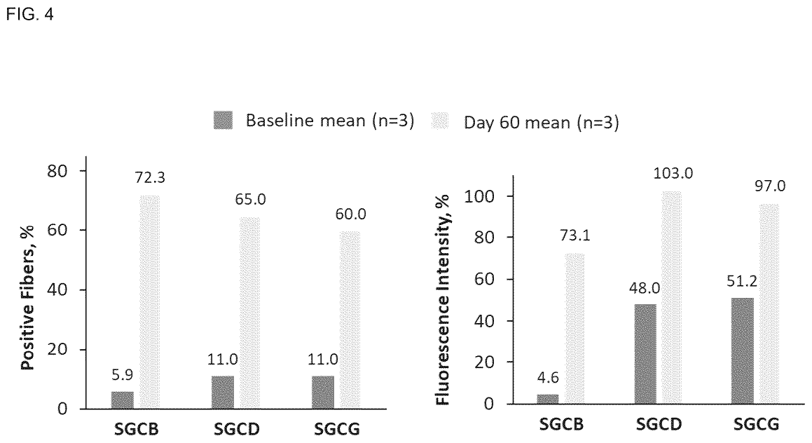

The pHELP adenovirus helper plasmid is 11,635 bp and was obtained from Applied Viromics. The plasmid contains the regions of adenovirus genome that are important for AAV replication, namely E2A, E40RF6, and VA RNA (the adenovirus E1 functions are provided by the 293 cells). The adenovirus sequences present in this plasmid only represents ˜40% of the adenovirus genome, and does not contain the cis elements critical for replication such as the adenovirus terminal repeats. Therefore, no infectious adenovirus is expected to be generated from such a production system. A schematic map of the pHELP plasmid is shown in FIG. 4.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In another embodiment, the disclosure contemplates compositions comprising rAAV of the present disclosure. Compositions of the disclosure comprise rAAV and a pharmaceutically acceptable carrier. The compositions may also comprise other ingredients such as diluents and adjuvants. Acceptable carriers, diluents and adjuvants are nontoxic to recipients and are preferably inert at the dosages and concentrations employed and include buffers and surfactants such as pluronics.

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg). One exemplary method of determining encapsilated vector genome titer uses quantitative PCR such as the methods described in (Pozsgai et al., Mol. Ther. 25(4): 855-869, 2017). Unless stated otherwise, the dosages described herein correspond to a dose as determined by the supercoiled DNA standard.

Methods of transducing a target cell with rAAV, in vivo or in vitro, are contemplated by the disclosure. The in vivo methods comprise the step of administering an effective dose, or effective multiple doses, of a composition comprising a rAAV of the disclosure to an animal (including a human being) in need thereof. If the dose is administered prior to development of a disorder/disease, the administration is prophylactic. If the dose is administered after the development of a disorder/disease, the administration is therapeutic. In embodiments of the disclosure, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the disclosure is muscular dystrophy, e.g. limb girdle muscular dystrophy or Duchenne muscular dystrophy.

Combination therapies are also contemplated by the disclosure. Combination as used herein includes both simultaneous treatment and sequential treatments. Combinations of methods of the disclosure with standard medical treatments (e.g., corticosteroids) are specifically contemplated, as are combinations with novel therapies.

Administration of an effective dose of the compositions, combination therapies or medicaments may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) that are to express the hSGCB protein.

The disclosure provides for local administration and systemic administration of an effective dose of rAAV, medicaments and compositions of the disclosure. For example, systemic administration is administration into the circulatory system so that the entire body is affected. Systemic administration includes enteral administration such as absorption through the gastrointestinal tract and parenteral administration through injection, infusion or implantation.

In particular, actual administration of rAAV of the present disclosure may be accomplished by using any physical method that will transport the rAAV recombinant vector into the target tissue of an animal. Administration according to the disclosure includes, but is not limited to, injection into muscle and injection into the bloodstream. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The rAAV can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical carriers, diluents or excipients suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the disclosure results in sustained expression of the hSGCB protein. The present disclosure thus provides methods of administering/delivering rAAV which express hSGCB protein to an animal, preferably a human being. These methods include transducing tissues (including, but not limited to, tissues such as muscle, organs such as liver and brain, and glands such as salivary glands) with one or more rAAV of the present disclosure. Transduction may be carried out with gene cassettes comprising tissue specific control elements. For example, one embodiment of the disclosure provides methods of transducing muscle cells and muscle tissues directed by muscle specific control elements, including, but not limited to, those derived from the actin and myosin gene families, such as from the myoD gene family (See Weintraub et al., Science, 251: 761-766 (1991)), the myocyte-specific enhancer binding factor MEF-2 (Cserjesi and Olson, Mol Cell Biol 11: 4854-4862 (1991)), control elements derived from the human skeletal actin gene (Muscat et al., Mol Cell Biol, 7: 4089-4099 (1987)), control elements derived from the cardiac actin gene, muscle creatine kinase sequence elements (See Johnson et al., Mol Cell Biol, 9:3393-3399 (1989)) and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factor binding element (Semenza et al., Proc Natl Acad Sci USA, 88: 5680-5684 (1991)), steroid-inducible elements and promoters including the glucocorticoid response element (GRE) (See Mader and White, Proc. Natl. Acad. Sci. USA 90: 5603-5607 (1993)), and other control elements.

Muscle tissue is an attractive target for in vivo DNA delivery, because it is not a vital organ and is easy to access. The disclosure contemplates sustained expression of hSGCB from transduced myofibers.

Thus, the disclosure provides methods of administering an effective dose (or doses, administered essentially simultaneously or doses given at intervals) of rAAV that encode hSGCB to a subject in need thereof.

Titers of rAAV to be administered in methods of the invention will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10³ to about 1×10¹⁴ or more DNase resistant particles (DRP) per ml. Dosages may also be expressed in units of viral genomes (vg). The titers of rAAV may be determined by the supercoiled plasmid quantitation standard or the linearized plasmid quantitation standard.

Methods of transducing a target cell with rAAV, in vivo or in vitro, are contemplated by the invention. The in vivo methods comprise the step of administering an effective dose, or effective multiple doses, of a composition comprising a rAAV of the invention to an animal (including a human being) in need thereof. If the dose is administered prior to development of a disorder/disease, the administration is prophylactic. If the dose is administered after the development of a disorder/disease, the administration is therapeutic. In embodiments of the invention, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the invention is muscular dystrophy, such as limb-girdle muscular dystrophy. Thus, provided is a method of tranducing a target cell with an rAAV scAAVrh74.MHCK7.hSGCB, which comprises a nucleotide sequence of SEQ ID NO: 1.

Combination therapies are also contemplated by the invention. Combination as used herein includes both simultaneous treatment or sequential treatments. Combinations of methods of the invention with standard medical treatments (e.g., steroids, corticosteroids, and/or glucocorticoids including but not limited to one or more of prednisone, prednisolone; and deflazacort) are specifically contemplated, as are combinations with novel therapies. In this regard, the combinations include administering to a subject one or more steroids, corticosteroids, and/or glucocorticoids including but not limited to one or more of prednisone, prednisolone; and deflazacort before administering an rAAV of the inventive methods to the subject, simultaneously with administering the rAAV to the subject, or after administering the rAAV to the subject.

In related embodiments of a combination therapy contemplated by the invention, the glucocorticoid includes, but is not limited to beclomethasone, betamethasone, budesonide, cortisone, dexamethasone, hydrocortisone, methylprednisolone, or triamcinolone.

It is recognized that an antigen specific T-cell response may occur in a subject administered with the rAAV vector. This is an expected response between 2-4 weeks following gene transfer. One possible consequence to such antigen specific T-cell responses is clearance of the transduced cells and loss of transgene expression. To dampen the host immune response to the rAAV based therapy, before the therapy, for example, twenty-four hours prior to the therapy procedure, subjects can be started on approximately 1 mg/kg/day prophylactic prednisone or comparable glucocorticoid by mouth with a maximum dose of 60 mg/day. IV administration of a comparable glucocorticoid at the approximate dose of 1 mg/kg/day would also be allowable if needed. Treatment will continue for approximately one month. A tapering protocol for prednisone or comparable glucocorticoid can be implemented based on individual subjects' immune response to the gene transfer, assessed by ELISpot assay and also by liver function monitoring with GGT.

A therapeutically effective amount of the rAAV vector is a dose of rAAV ranging from about 1e13 vg/kg to about 5e14 vg/kg, or about 1e13 vg/kg to about 2e13 vg/kg, or about 1e13 vg/kg to about 3e13 vg/kg, or about 1e13 vg/kg to about 4e13 vg/kg, or about 1e13 vg/kg to about 5e13 vg/kg, or about 1e13 vg/kg to about 6e13 vg/kg, or about 1e13 vg/kg to about 7e13 vg/kg, or about 1e13 vg/kg to about 8e13 vg/kg, or about 1e13 vg/kg to about 9e13 vg/kg, or about 1e13 vg/kg to about 1e14 vg/kg, or about 1e13 vg/kg to about 2e14 vg/kg, or 1e13 vg/kg to about 3e14 vg/kg, or about 1e13 to about 4e14 vg/kg, or about 3e13 vg/kg to about 4e13 vg/kg, or about 3e13 vg/kg to about 5e13 vg/kg, or about 3e13 vg/kg to about 6e13 vg/kg, or about 3e13 vg/kg to about 7e13 vg/kg, or about 3e13 vg/kg to about 8e13 vg/kg, or about 3e13 vg/kg to about 9e13 vg/kg, or about 3e13 vg/kg to about 1e14 vg/kg, or about 3e13 vg/kg to about 2e14 vg/kg, or 3e13 vg/kg to about 3e14 vg/kg, or about 3e13 to about 4e14 vg/kg, or about 3e13 vg/kg to about 5e14 vg/kg, or about 5e13 vg/kg to about 6e13 vg/kg, or about 5e13 vg/kg to about 7e13 vg/kg, or about 5e13 vg/kg to about 8e13 vg/kg, or about 5e13 vg/kg to about 9e13 vg/kg, or about 5e13 vg/kg to about 1e14 vg/kg, or about 5e13 vg/kg to about 2e14 vg/kg, or 5e13 vg/kg to about 3e14 vg/kg, or about 5e13 to about 4e14 vg/kg, or about 5e13 vg/kg to about 5e14 vg/kg, or about 1e14 vg/kg to about 2e14 vg/kg, or 1e14 vg/kg to about 3e14 vg/kg, or about 1e14 to about 4e14 vg/kg, or about 1e14 vg/kg to about 5e14 vg/kg, 6e14 vg/kg, 7e14 vg/kg, 8e14 vg/kg, or 9e14 vg/kg. The invention also comprises compositions comprising these ranges of rAAV vector.

For example, a therapeutically effective amount of rAAV vector is a dose of 1e13 vg/kg, about 2e13 vg/kg, about 3e13 vg/kg, about 4e13 vg/kg, about 5e13 vg/kg, about 6e13 vg/kg, about 7e13 vg/kg, about 7.4e13 vg/kg,about 8e13 vg/kg, about 9e13 vg/kg, about 1e14 vg/kg, about 2e14 vg/kg, about 3e14 vg/kg, about 4e14 vg/kg and 5e14 vg/kg. The titer or dosage of AAV vectors can vary based on the physical forms of plasmid DNA as a quantitation standard. For example, the value of titer or dosage may vary based off of a supercoiled standard qPCR titering method or a linear standard qPCR tittering method. In one embodiment, a therapeutically effective amount of rAAV is a dose of 5e13 vg/kg based on a supercoiled plasmid as the quantitation standard or a dose of 1.85e13 vg/kg based on a linearized plasmid as the quantitation standard. In another embodiment, a therapeutically effective amount of rAAV is a dose of 2e14 vg/kg based on the supercoiled plasmid as the quantitation standard or a dose of 7.41e13 vg/kg based on the linearized plasmid as the quantitation standard. In another embodiment, the therapeutically effective amount of scAAVrh74.MHCK7.hSGCB is a dose ranging from about 1e13 vg/kg to about 5e14 vg/kg, or about 1e13 vg/kg to about 2e13 vg/kg, or about 1e13 vg/kg to about 3e13 vg/kg, or about 1e13 vg/kg to about 4e13 vg/kg, or about 1e13 vg/kg to about 5e13 vg/kg, or about 1e13 vg/kg to about 6e13 vg/kg, or about 1e13 vg/kg to about 7e13 vg/kg, or about 1e13 vg/kg to about 8e13 vg/kg, or about 1e13 vg/kg to about 9e13 vg/kg, or about 1e13 vg/kg to about 1e14 vg/kg, or about 1e13 vg/kg to about 2e14 vg/kg, or 1e13 vg/kg to about 3e14 vg/kg, or about 1e13 to about 4e14 vg/kg, or about 3e13 vg/kg to about 4e13 vg/kg, or about 3e13 vg/kg to about 5e13 vg/kg, or about 3e13 vg/kg to about 6e13 vg/kg, or about 3e13 vg/kg to about 7e13 vg/kg, or about 3e13 vg/kg to about 8e13 vg/kg, or about 3e13 vg/kg to about 9e13 vg/kg, or about 3e13 vg/kg to about 1e14 vg/kg, or about 3e13 vg/kg to about 2e14 vg/kg, or 3e13 vg/kg to about 3e14 vg/kg, or about 3e13 to about 4e14 vg/kg, or about 3e13 vg/kg to about 5e14 vg/kg, or about 5e13 vg/kg to about 6e13 vg/kg, or about 5e13 vg/kg to about 7e13 vg/kg, or about 5e13 vg/kg to about 8e13 vg/kg, or about 5e13 vg/kg to about 9e13 vg/kg, or about 5e13 vg/kg to about 1e14 vg/kg, or about 5e13 vg/kg to about 2e14 vg/kg, or 5e13 vg/kg to about 3e14 vg/kg, or about 5e13 to about 4e14 vg/kg, or about 5e13 vg/kg to about 5e14 vg/kg, or about 1e14 vg/kg to about 2e14 vg/kg, or 1e14 vg/kg to about 3e14 vg/kg, or about 1e14 to about 4e14 vg/kg, or about 1e14 vg/kg to about 5e14 vg/kg, 6e14 vg/kg, 7e14 vg/kg, 8e14 vg/kg, or 9e14 vg/kg, based on the supercoiled plasmid as the quantitation standard. The invention also comprises compositions comprising these doses of rAAV vector.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal.

Route(s) of administration and serotype(s) of AAV components of the rAAV (in particular, the AAV ITRs and capsid protein) of the invention may be chosen and/or matched by those skilled in the art taking into account the infection and/or disease state being treated and the target cells/tissue(s) that are to express the β-sarcoglycan.

The invention provides for local administration and systemic administration of an effective dose of rAAV and compositions of the invention. For example, systemic administration is administration into the circulatory system so that the entire body is affected. Systemic administration includes enteral administration such as absorption through the gastrointestinal tract and parental administration through injection, infusion or implantation.

In particular, actual administration of rAAV of the present invention may be accomplished by using any physical method that will transport the rAAV recombinant vector into the target tissue of an animal. Administration according to the invention includes, but is not limited to, injection into muscle, the bloodstream and/or directly into the liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein.

Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The rAAV can be used with any pharmaceutically acceptable carrier for ease of administration and handling. Thus, in another aspect, the application is directed to a formulation that comprises an rAAV that comprises an AAVrh74 derived capsid, a buffer agent, an ionic strength agent, and a surfactant. In one embodiment, the rAAV is at a concentration of about 1.0×10¹² vg/ml to about 5.0×10¹⁴ vg/ml. In another embodiment, the rAAV is at a concentration of about 5.0×10¹² vg/ml to about 1.0×10¹⁴ vg/ml based on a supercoiled plasmid as the quantitation standard. In another embodiment, the rAAV is at a concentration of about 2.0×10¹³ vg/ml based on a supercoiled plasmid as the quantitation standard. In one embodiment, the rAAV is an scAAVrh74.MHCK7.hSGCB vector. In one embodiment, the concentration of rAAV in the composition or formulation is from 1×10¹³ vg/ml to 2×10¹⁴ vg/ml based on a supercoiled plasmid as the quantitation standard. In another embodiment, the concentration is 2×10¹³ vg/ml, 4×10¹³ vg/ml, or 5×10¹³ vg/ml based on a supercoiled plasmid as the quantitation standard. In one embodiment, the buffer agent comprises one or more of tris, tricine, Bis-tricine, HEPES, MOPS, TES, TAPS, PIPES, and CAPS. In another embodiment, the buffer agent comprises tris with pH 8.0 at concentration of about 5 mM to about 40 mM. In one embodiment, the buffer agent comprises tris with pH 8.0 at about 20 mM. In one embodiment, the ionic strength agent comprises one of more of potassium chloride (KCl), potassium acetate, potassium sulfate, ammonium sulfate, ammonium chloride (NH₄Cl), ammonium acetate, magnesium chloride (MgCl₂), magnesium acetate, magnesium sulfate, manganese chloride (MnCl2), manganese acetate, manganese sulfate, sodium chloride (NaCl), sodium acetate, lithium chloride (LiCl), and lithium acetate. In one embodiment, the ionic strength agent comprises MgCl₂ at a concentration of about 0.2 mM to about 4 mM. In another embodiment, the ionic strength agent comprises NaCl at a concentration of about 50 mM to about 500 mM. In another embodiment, the ionic strength agent comprises MgCl₂ at a concentration of about 0.2 mM to about 4 mM and NaCl at a concentration of about 50 mM to about 500 mM. In another embodiment, the ionic strength agent comprises MgCl₂ at a concentration of about 1 mM and NaCl at a concentration of about 200 mM. In one embodiment, the surfactant comprises one or more of a sulfonate, a sulfate, a phosphonate, a phosphate, a Poloxamer, and a cationic surfactant. In one embodiment, the Poloxamer comprises one or more of Poloxamer 124, Poloxamer 181, Poloxamer 184, Poloxamer 188, Poloxamer 237, Poloxamer 331, Poloxamer 338, and Poloxamer 407. In one embodiment, the surfactant comprises the Poloxamer at a concentration of about 0.00001% to about 1%. In another embodiment, the surfactant comprises Poloxamer 188 at a concentration of about 0.001%. For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

Thus, also described herein are methods of administering an effective dose (or doses, administered essentially simultaneously or doses given at intervals) of rAAV that encode β-sarcoglycan to a mammalian subject in need thereof.

All publications and patents mentioned herein are hereby incorporated by reference in their entirety as if each individual publication or patent was specifically and individually indicated to be incorporated by reference. In case of conflict, the present application, including any definitions herein, will control.

The invention is further described in the following Examples, which do not limit the scope of the invention described in the claims.

### EXAMPLES

Preclinical studies using scAAVrh74.MHCK7.hSGCB are described in International Patent Publication No. WO 2017/180976, which is incorporated by reference herein in its entirety.

### Example 1

### scAAVrh.74.MHCK7.hSGCB Construction

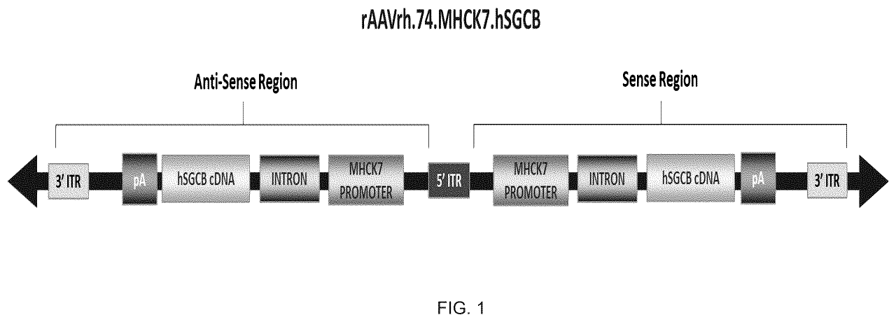

The transgene cassette containing a codon-optimized full-length human SGCB cDNA as shown in FIG. 1 was constructed. The cassette includes a consensus Kozak sequence (CCACC), an SV40 chimeric intron, a synthetic polyadenylation site, and the muscle-specific MHCK7 used to drive expression of the cassette. This is an MCK based promoter which utilizes a 206-bp enhancer taken from −1.2 kb 5′ of the transcription start site within the endogenous muscle creatine kinase gene with a proximal promoter (enh358MCK, 584-bp). The cassette was packaged into a self-complementary (sc) AAVrh.74 vector that is 93% homologous to AAV8. AAVrh.74 has been shown in mice and non-human primates to be safe and effective, particularly in crossing the vascular barrier when delivered to muscle through the circulation. (Chicoine et al., Mol Ther 2014; 22: 713-724, Rodino-Klapac et al., Mol Ther 2010; 18: 109-117, Chicoine et al., Mol Ther 2014; 22: 338-347)

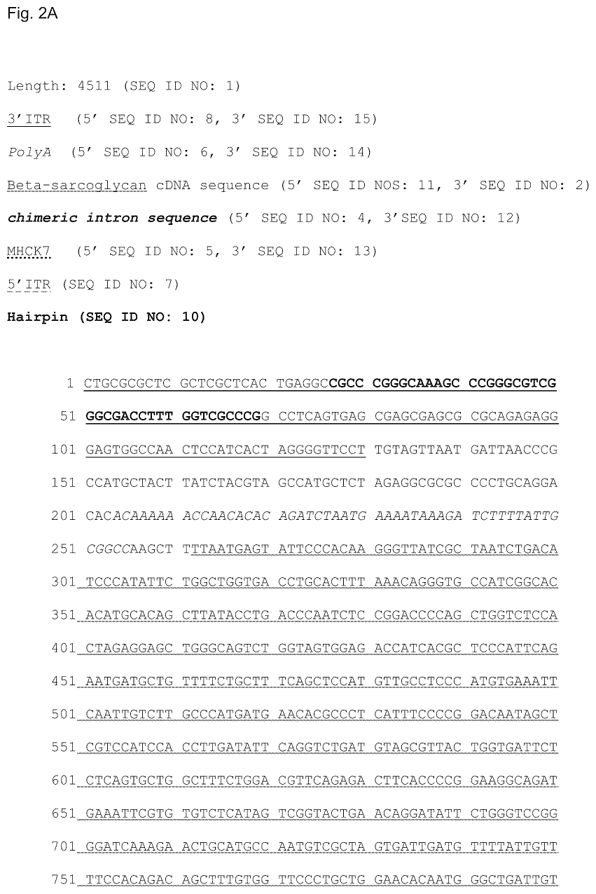

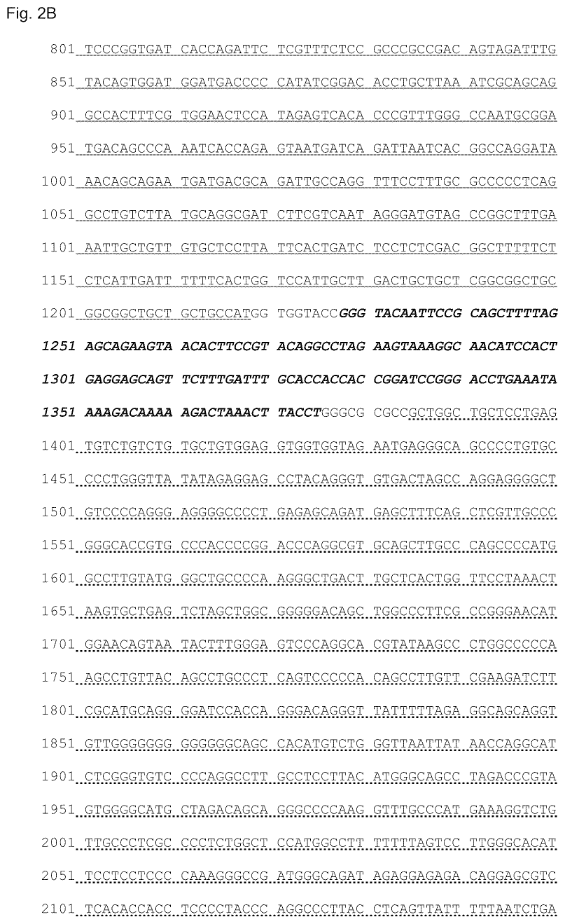

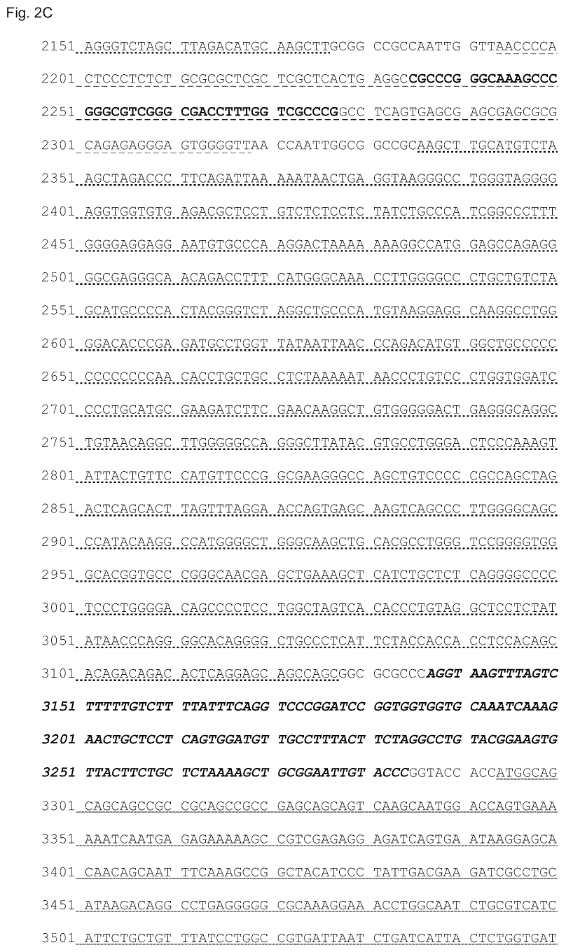

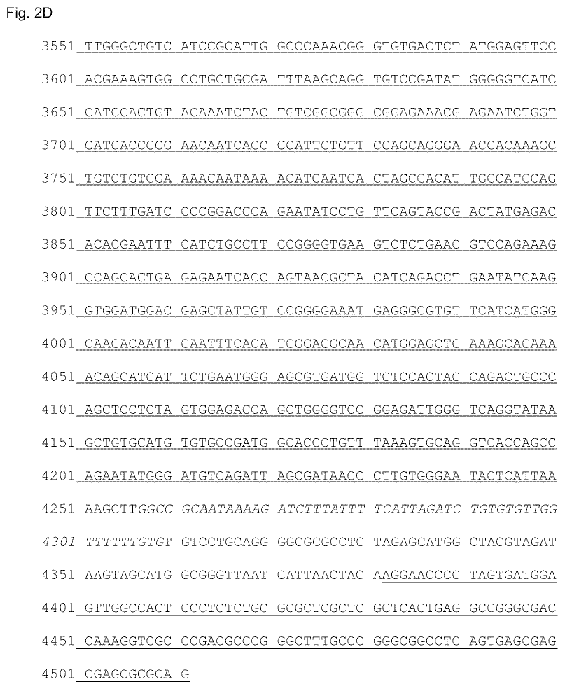

A single strand AAV vector (ssAAV), once in the nucleus, requires cell-mediated synthesis of the second strand before it is ready for replication and transcription. An exemplary self-complementary AAV vector (scAAV) has the structure as set out in FIG. 1 and the annotated nucleotide sequence provided in FIG. 2 and is described in Table below. This scAAV is superior to ssAAV in gene therapy because the scAAV bypasses the rate-limiting step of the cellular synthesis of the second-strand as required in a ssAAV.

Molecular Features of scAAVrh.74.MHCK7.hSGCB

| TYPE | START | END | NAME | DESCRIPTION |
|---|---|---|---|---|
| REGION | 1 | 130 | 3′ ITR | Self-complementary sequence 3′ |
| | | | | inverted terminal repeat |
| REGION | 27 | 69 | Hairpin | Hairpin within the 3′ ITR |
| REGION | 204 | 255 | PolyA | Self-complementary |
| | | | | sequence polyA |
| GENE | 262 | 1218 | hSCGB | Self-complementary hSCGB |
| | | | cDNA | cDNA |
| REGION | 1228 | 1375 | Chimeric | 5′ donor site from |
| | | | intron | human β-globin |
| | | | | gene and the branchpoint |
| | | | | and 3′ splice acceptor site |
| | | | | from IgG heavy chain |
| | | | | variable region |
| REGION | 1385 | 2176 | MHCK7 | Self-complementary |
| | | | | mouse myosin heavy chain |
| | | | | complex-E box muscle |
| | | | | creatine kinase fusion |
| | | | | enhancer/promoter |
| REGION | 2194 | 2318 | 5′ ITR | 5′ ITR |
| GENE | 2336 | 3127 | MHCK7 | Self-complementary |
| | | | | mouse myosin heavy |
| | | | | chain complex-E box muscle |
| | | | | creatine kinase fusion |
| | | | | enhancer/promoter |
| REGION | 3137 | 3284 | Chimeric | 5′ donor site from |
| | | | intron | human β-globin |
| | | | | gene and the branchpoint |
| | | | | and 3′ splice acceptor site |
| | | | | from IgG heavy chain |
| | | | | variable region |
| GENE | 3294 | 4250 | hSCGB | hSCGB cDNA |
| | | | cDNA | |
| REGION | 4257 | 4309 | PolyA | PolyA |
| REGION | 4382 | 4511 | 3′ ITR | 3′ ITR |

### Example 2

### LGMD2E Open-label Trial

Six patients treated with rAAVrh74.MHCK7.SGCB demonstrated robust target tissue transduction and transgene expression (biceps, TA) (FIG. 3), as well as clinical efficacy with improvement in multiple ambulatory clinical outcome measures. In this clinical dose-escalation study, two dose cohorts (each with n=3 patients) were evaluated where patients received rAAVrh74.MHCK7.SGCB (denoted to herein as “SRP-9003”) at a dose of 1.85×10¹³ vg/kg and 7.41×10¹³ vg/kg via intravenous infusion. The dosage of 1.85×10¹³ vg/kg was measured by qPCR using linear reference plasmid DNA qPCR with its supercoiled reference DNA equivalent at 5×10¹³ vg/kg. The 7.41×10¹³ vg/kg was measured by qPCR using linear reference plasmid DNA qPCR with its supercoiled reference DNA equivalent at 2×10¹⁴ vg/kg.

As presented in Table 1, dose-dependent increase in both target tissue transduction (measured as vector genome copies per nucleus) and percent of β-SG protein (measured as immunofluorescence of mean percent β-sarcoglycan positive fibers (POSGPF), and immunofluorescence of mean percent fluorescent expression (PFE), and total protein expression by Western Blot) was observed. Furthermore, in Cohort 1 patients treated with 1.84×10¹³ vg/kg for whom the Year 2 biopsy has recently been obtained, the expression of β-SG was sustained at high levels between Day 60 and Year 2.

TABLE 1
Biological Activity of rAAVrh74.MHCK7.SGCB at Day 60 and Year 2 Post-
Transfer as Measured by qPCR, Immunofluorescence, and Western Blot Methods

| Cohort | Cohort 1 (1.85 × 10¹³ vg/kg) | | | | Cohort 2 (7.41 × 10¹³ vg/kg) | | | |
|---|---|---|---|---|---|---|---|---|
| Patient No. | E01 | E02 | E03 | Mean | E04 | E05 | E07 | Mean |

Day 60 gene expression assessments

| | | | | | | | | |
|---|---|---|---|---|---|---|---|---|
| Average vg | 1.05 | 0.44 | 0.27 | 0.60 | 4.02 | 7.15 | 1.57 | 4.2 |
| copies/nucleus | | | | | | | | |
| Immunofluorescence: | 63% | 49% | 42% | 51% | 65% | 77% | 75% | 72% |
| mean PβSGPF | | | | | | | | |
| Immunofluorescence: | 47% | 57% | 38% | 47% | 55% | 67% | 97% | 73% |
| mean PFE | | | | | | | | |
| Western blot: | 35% | 39% | 35% | 36% | 53% | 63% | 70% | 62% |
| % normal ª | | | | | | | | |

Year 2 gene expression assessments

| | | | | | | | | |
|---|---|---|---|---|---|---|---|---|
| Average vg | 0.12 | 0.08 | 0.20 | 0.13 | — | — | — | — |
| copies/nucleus | | | | | | | | |
| Immunofluorescence: | 22% | 61% | 60% | 48% | — | — | — | — |
| mean PβSGPF | | | | | | | | |
| Immunofluorescence: | 9% | 51% | 46% | 35% | — | — | — | — |
| mean PFE | | | | | | | | |
| Western blot: | 37% | 69% | 56% | 54% | — | — | — | — |
| % normal ^{a} | | | | | | | | |

PβSGPF = percent β-sarcoglycan positive fibers;
PFE = percent fluorescent expression;
qPCR = quantitative polymerase chain reaction;
vg = vector genome.
Data include tibialis anterior and biceps.
^{a} Presented as percentage of normal. Normal control defined as the average wild-type β-sarcoglycan expression (n = 3).

A deficiency of beta-sarcoglycan (SGCB) protein leads to reduction or complete loss of the other sarcoglycan subunits from the membrane. Following administration of the SRP-9003, reconstitution of SGCB expression led to an increase in 6-sarcoglycan (SGCD) and 7-sarcoglycan (SGCG) expression (FIG. 4). The concomitant increases in expression of other sarcoglycans and localization of 3-SG to the sarcolemmal membrane (as measured by percent SGCB positive fiber and fiber intensity) shows that the SGCB expression induced by rAAVrh74.IVIICK7. SGCB was functional and localized correctly within the myocyte as would be expected for the native SGCB protein.

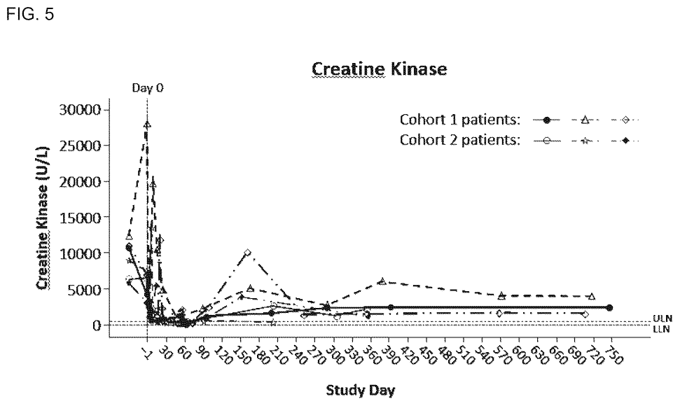

Elevated CK levels are inversely and statistically significantly correlated with disease duration (Semplicini 2015), implying that marked elevations in CK, such as those observed at Baseline in patients in Cohort 1, connote active processes of muscle deterioration (i.e., active disease prior to permanent deterioration). Systemic gene transfer with SRP-9003 led to early and deep reductions in mean CK levels, which were sustained (74%) at the day 750 assessments (FIG. 5).

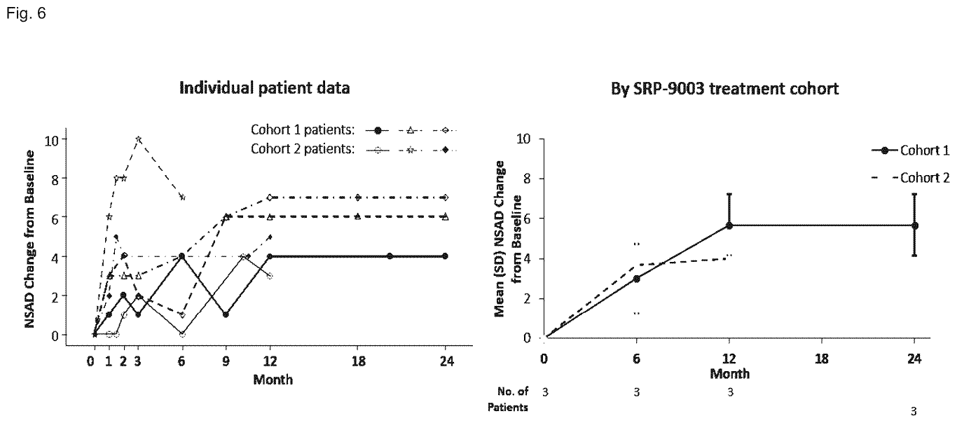

Patients who were treated with rAAVrh74.MHCK7.SGCB demonstrated clinically meaningful improvement in multiple functional motor assessments inclusive of North Star Assessment for Dysferlinopathy (NSAD) and timed function tests during Year 1 and Year 2 (FIG. 6 and Table 2). Functional improvements in both cohorts were most impressively noted by the overall 5 point improvements across the 2 dose cohorts in the total NSAD scores. Importantly, the improvements observed in the treated patients were sustained at the end of Year 2.

TABLE 2
Improvements over baseline in timed function tests

| Mean change | Cohort 1 | | | Cohort 2 | |
|---|---|---|---|---|---|
| from | (1.85 × 10¹³ vg/kg) | | | (7.41 × 10¹³ vg/kg) | |
| baseline | | 12 | 24 | 6 | 12 |
| (sec) | 6 months | months | months | months | months |
| Time to rise | −0.2 | −0.8 | −0.6 | −1.3 | −1.1 |
| 4-stair climb | −0.5 | −0.5 | −0.3 | −0.4 | −0.4 |
| 100 m | −3.8 | −5.3 | −2.8 | −6.3 | −7.9 |
| 10 m | −0.6 | −0.6 | −0.2 | −0.6 | −0.6 |

### Example 3

### Natural History Study

To select patients in the “natural history” control cohort for comparison with the treated patients in rAAVrh74.MHCK7.SGCB study, the following criteria, the same as the pre-defined inclusion criteria for the open label clinical study, were applied to the “natural history” dataset:
Restricted to only LGMD2E/R4 ambulatory patients. For this cohort, it was defined as those with non-missing 10MWR value.
Baseline age 4 through 15 years, inclusive
100MWR test result: ≥40% of that predicted for age-, height-, gender-, and weight matched healthy controls

With respect to the criteria applied to the overall “natural history” database in order to derive a natural history comparator for rAAVrh74.MHCK7.SGCB (SRP-9003) treated patients, from an overall dataset of 35 patients, 5 control patients who met these criteria were identified. The baselines between the treated cohort and natural history cohort are compared in Table 3. Age and gender are well balanced between rAAVrh74.MHCK7.SGCB treated subjects and “natural history” control cohort. Notably, baseline functional endpoints scores are higher in NCH control cohort.

TABLE 3
Baseline comparison of SRP-9003-treated patients with
natural history cohort

| | SRP-9003-101 | NCH |
|---|---|---|
| | (N = 6) | (N = 5) |
| Male, n (%) | 3 (50) | 3 (60) |
| Age (years) | 10.0 (3.5) | 9.8 (3.2) |
| NSAD | 41.2 (3.7) | 49.0 (3.9) |
| 100 m (sec) | 51.4 (10.5) | 38.9 (3.9) |
| 10 m (sec) | 5.1 (0.9) | 4.4 (0.3)^{a} |

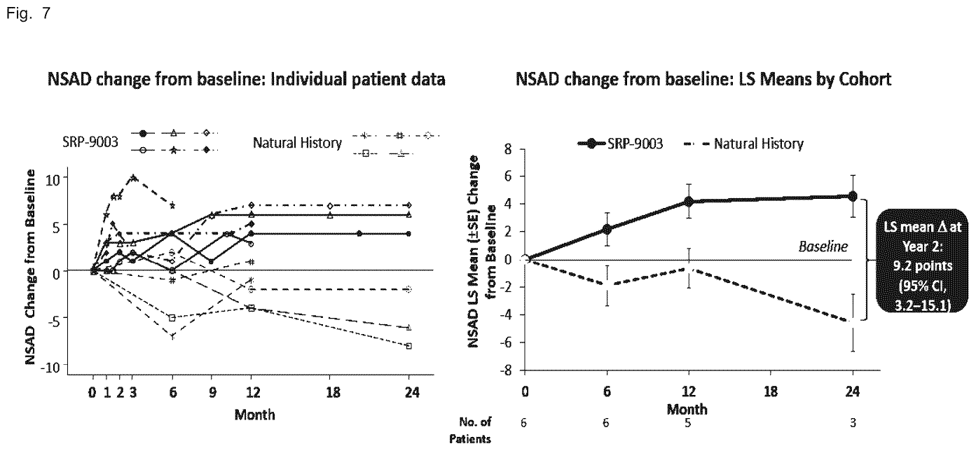

Following dosing of rAAVrh74.MHCK7.SGCB, an overall improvement was observed in the gene therapy treated subjects, compared to a downward trend in the “natural history” control cohort (FIG. 7). Individual subjects' change from baseline in NSAD scores are provided in NH=natural history; NSAD=North Star Assessment for Dysferlinopathy; SD=standard deviation. Patients treated with SRP-9003 demonstrated clinically meaningful improvements in functional outcomes in an exploratory comparison to an LGMD2E/R4 natural history cohort, as measured by NSAD.

The safety and tolerability of rAAVrh74.MHCK7.SGCB were determined by assessing adverse events, physical examinations, vital signs, serum and urine laboratory results, and immunogenicity at every visit through Month 18 for Cohort 1 and Month 6 for Cohort 2. In both cohorts, no other laboratory abnormalities were suggestive of safety concerns. No decreases in platelet counts were observed outside of the normal range. No clinical sequelae were associated with complement activation. Results show no new safety signals, and all treatment-related AEs occurred early and were transient and manageable.

### REFERENCES

1 Bonnemann C G, Modi R, Noguchi S, Mizuno Y, Yoshida M, Gussoni E et al. Beta-sarcoglycan (A3b) mutations cause autosomal recessive muscular dystrophy with loss of the sarcoglycan complex. Nat Genet 1995; 11: 266-273.
2 Moore S A, Shilling C J, Westra S, Wall C, Wicklund M P, Stolle C et al. Limb-girdle muscular dystrophy in the United States. J Neuropathol Exp Neurol 2006; 65: 995-1003.
3 Araishi K, Sasaoka T, Imamura M, Noguchi S, Hama H, Wakabayashi E et al. Loss of the sarcoglycan complex and sarcospan leads to muscular dystrophy in beta-sarcoglycan-deficient mice. Hum Mol Genet 1999; 8: 1589-1598.
4 Durbeej M, Cohn R D, Hrstka R F, Moore S A, Allamand V, Davidson B L et al. Disruption of the beta-sarcoglycan gene reveals pathogenetic complexity of limb-girdle muscular dystrophy type 2E. Mol Cell 2000; 5: 141-151.
5 Bonnemann C G, Passos-Bueno M R, McNally E M, Vainzof M, de Sa Moreira E, Marie S K et al. Genomic screening for beta-sarcoglycan gene mutations: missense mutations may cause severe limb-girdle muscular dystrophy type 2E (LGMD 2E). Hum Mol Genet 1996; 5: 1953-1961.
6 Angelini C, Fanin M, Freda M P, Duggan D J, Siciliano G, Hoffman E P. The clinical spectrum of sarcoglycanopathies. Neurology 1999; 52: 176-179.
7 Sandona D, Betto R. Sarcoglycanopathies: molecular pathogenesis and therapeutic prospects. Exp Rev Mol Med 2009; 11: e28.
8 Fanin M, Melacini P, Boito C, Pegoraro E, Angelini C. LGMD2E patients risk developing dilated cardiomyopathy. Neuromusc Disord 2003; 13: 303-309.
9 Sveen M L, Thune J J, Kober L, Vissing J. Cardiac involvement in patients with limb-girdle muscular dystrophy type 2 and Becker muscular dystrophy. Arch Neurol 2008; 65: 1196-1201.
10 Melacini P, Fanin M, Duggan D J, Freda M P, Berardinelli A, Danieli G A et al. Heart involvement in muscular dystrophies due to sarcoglycan gene mutations. Muscle Nerve 1999; 22: 473-479.
11 Narayanaswami P, Weiss M, Selcen D, David W, Raynor E, Carter G et al. Evidence-based guideline summary: diagnosis and treatment of limb-girdle and distal dystrophies: report of the guideline development subcommittee of the American Academy of Neurology and the practice issues review panel of the American Association of Neuromuscular & Electrodiagnostic Medicine. Neurology 2014; 83: 1453-1463.
12 Wong-Kisiel L C, Kuntz N L. Two siblings with limb-girdle muscular dystrophy type 2E responsive to deflazacort. Neuromusc Disord 2010; 20: 122-124.
13 Barresi R, Di Blasi C, Negri T, Brugnoni R, Vitali A, Felisari G et al. Disruption of heart sarcoglycan complex and severe cardiomyopathy caused by beta sarcoglycan mutations. J Med Genet 2000; 37: 102-107.
14 Gibertini S, Zanotti S, Savadori P, Curcio M, Saredi S, Salerno F et al. Fibrosis and inflammation are greater in muscles of beta-sarcoglycan-null mouse than mdx mouse. Cell Tissue Res 2014; 356: 427-443.
15 McCarty D M, Fu H, Monahan P E, Toulson C E, Naik P, Samulski R J. Adeno-associated virus terminal repeat (T R) mutant generates self-complementary vectors to overcome the rate-limiting step to transduction in vivo. Gene Ther 2003; 10: 2112-2118.
16 McCarty D M, Monahan P E, Samulski R J. Self-complementary recombinant adeno-associated virus (scAAV) vectors promote efficient transduction independently of DNA synthesis. Gene Ther 2001; 8: 1248-1254.
17 Chicoine L G, Rodino-Klapac L R, Shao G, Xu R, Bremer W G, Camboni M et al. Vascular delivery of rAAVrh74.MCK.GALGT2 to the gastrocnemius muscle of the rhesus macaque stimulates the expression of dystrophin and laminin alpha2 surrogates. Mol Ther 2014; 22: 713-724.
18 Rodino-Klapac L R, Montgomery C L, Bremer W G, Shontz K M, Malik V, Davis N et al. Persistent expression of FLAG-tagged micro dystrophin in nonhuman primates following intramuscular and vascular delivery. Mol Ther 2010; 18: 109-117.
19 Rodino-Klapac L R, Janssen P M, Montgomery C L, Coley B D, Chicoine L G, Clark K R et al. A translational approach for limb vascular delivery of the micro-dystrophin gene without high volume or high pressure for treatment of Duchenne muscular dystrophy. J Transl Med 2007; 5: 45.
20 Wang B, Li J, Fu F H, Chen C, Zhu X, Zhou L et al. Construction and analysis of compact muscle-specific promoters for AAV vectors. Gene Ther 2008; 15: 1489-1499.
21 Chicoine L G, Montgomery C L, Bremer W G, Shontz K M, Griffin D A, Heller K N et al. Plasmapheresis eliminates the negative impact of AAV antibodies on micro-dystrophin gene expression following vascular delivery. Mol Ther 2014; 22: 338-347.
22 Matsuda R, Nishikawa A, Tanaka H. Visualization of dystrophic muscle fibers in mdx mouse by vital staining with Evans blue: evidence of apoptosis in dystrophin-deficient muscle. J Biochem 1995; 118: 959-964.
23 Straub V, Rafael J A, Chamberlain J S, Campbell K P. Animal models for muscular dystrophy show different patterns of sarcolemmal disruption. J Cell Biol 1997; 139: 375-385.
24 Mendell J R, Sahenk Z, Malik V, Gomez A M, Flanigan K M, Lowes L P et al. A phase 1/2a follistatin gene therapy trial for becker muscular dystrophy. Mol Ther 2015; 23: 192-201.
25 Dressman D, Araishi K, Imamura M, Sasaoka T, Liu L A, Engvall E et al. Delivery of alpha- and beta-sarcoglycan by recombinant adeno-associated virus: efficient rescue of muscle, but differential toxicity. Hum Gene Ther 2002; 13: 1631-1646.
26 Rodino-Klapac L R, Lee J S, Mulligan R C, Clark K R, Mendell J R. Lack of toxicity of alpha-sarcoglycan overexpression supports clinical gene transfer trial in LGMD2D. Neurology 2008; 71: 240-247.
27 Shield M A, Haugen H S, Clegg C H, Hauschka S D. E-box sites and a proximal reg-ulatory region of the muscle creatine kinase gene differentially regulate expres¬sion in diverse skeletal muscles and cardiac muscle of transgenic mice. Mol Cell Biol 1996; 16: 5058-5068.
28 Rabinowitz J E, Rolling F, Li C, Conrath H, Xiao W, Xiao X et al. Cross-packaging of a single adeno-associated virus (AAV) type 2 vector genome into multiple AAV serotypes enables transduction with broad specificity. J Virol 2002; 76: 791-801.
29 Grieger J C, Choi V W, Samulski R J. Production and characterization of adeno-associated viral vectors. Nat Protoc 2006; 1: 1412-1428.
30 Clark K R, Liu X, McGrath J P, Johnson P R. Highly purified recombinant adeno-associated virus vectors are biologically active and free of detectable helper and wild-type viruses. Hum Gene Ther 1999; 10: 1031-1039.
31 Liu M, Yue Y, Harper S Q, Grange R W, Chamberlain J S, Duan D. Adeno-associated virus-mediated microdystrophin expression protects young mdx muscle from contraction-induced injury. Mol Ther 2005; 11: 245-256.
32 Hakim C H, Grange R W, Duan D. The passive mechanical properties of the extensor digitorum longus muscle are compromised in 2- to 20-mo-old mdx mice. J Appl Physiol 2011; 110: 1656-1663.
33 Wein N, Vulin A, Falzarano M S, Szigyarto C A, Maiti B, Findlay A et al. Translation from a DMD exon 5 IRES results in a functional dystrophin isoform that attenuates dystrophinopathy in humans and mice. Nat Med 2014; 20: 992-1000.

What is claimed is:

1. A recombinant AAV (rAAV) comprising a polynucleotide sequence wherein the polynucleotide sequence comprises i) two complementary nucleotide sequences, each encoding a gene of interest, wherein the two complementary nucleotide sequences encoding the gene of interest flank one central 5′ ITR sequence, ii) two complementary polyadenylation sequences,
wherein the polynucleotide sequence is flanked by a pair of two 3′ ITR sequences, wherein the two 3′ ITR sequences of the pair are complementary to each other, wherein the gene of interest encodes a protein of R sarcoglycan, a sarcoglycan, or 7 sarcoglycan.

2. The recombinant AAV of claim 1, wherein the 5′ ITR sequence comprises SEQ ID NO: 7.

3. The recombinant AAV of claim 1, wherein the pair of 3′ ITR sequences comprise SEQ ID NOs: 8 and 15.

4. The recombinant AAV of claim 1, wherein the gene of interest encodes a sequence as shown by SEQ ID NO: 3.

5. The recombinant AAV of claim 1, comprising SEQ ID NO: 1.

6. A recombinant AAV (rAAV) vector comprising a polynucleotide sequence, wherein the polynucleotide sequence comprises, from 5′ to 3′ direction,
(1) a complementary sequence of a polyadenylation sequence;
(2) a complementary sequence of a gene of interest;
(3) a complementary sequence of an intron;
(4) a complementary sequence of a promoter;
(5) a 5′ ITR sequence;
(6) the promoter;
(7) the intron;
(8) the gene of interest; and
(9) the polyadenylation sequence;
wherein the polynucleotide sequence is flanked by a pair of two 3′ ITR sequences, wherein the two 3′ ITR sequences of the pair are complementary to each other.

7. The rAAV vector of claim 6, wherein the gene of interest comprises human sarcoglycan-β (hSCGB), human sarchoglycan γ (hSCGG), human dysferlin, and human ANO5. Calpain-3 (Cap 3) gene.

8. The rAAV vector of claim 6, wherein the promoter is a muscle specific control element, wherein the muscle specific control element comprises human skeletal actin gene element, cardiac actin gene element, myocyte-specific enhancer binding factor mef, muscle creatine kinase (MCK), truncated MCK (tMCK), myosin heavy chain (MHC), MHCK7, C5-12, murine creatine kinase enhancer element, skeletal fast-twitch troponin c gene element, slow-twitch cardiac troponin c gene element, slow-twitch troponin i gene element, hypoxia-inducible nuclear factor binding element, or steroid-inducible element or glucocorticoid response element (gre).

9. The recombinant AAV of claim 6, wherein the 5′ ITR sequence comprises SEQ ID NO: 7.

10. The recombinant AAV of claim 6, wherein the 3′ ITR sequences of the pair comprise SEQ ID NOs: 8 and 15.

11. The recombinant AAV of claim 6, wherein the intron and the complementary sequence of the intron comprise SEQ ID NOs: 4 and 11, respectively.

12. The recombinant AAV of claim 6, wherein the gene of interest encodes a sequence as shown by SEQ ID NO: 3.

13. The recombinant AAV of claim 6, wherein the gene of interest and the complementary sequence of the gene of interest comprise SEQ ID NOs: 2 and 11, respectively.

14. The recombinant AAV of claim 6, wherein the promoter and the complementary sequence of the promoter comprise SEQ ID NO: 5 and 13, respectively.

15. The recombinant AAV of claim 6, wherein the polyadenylation sequence and the complementary sequence of the polyadenylation sequence comprise SEQ ID NO: 6 and 14, respectively.

16. The recombinant AAV of claim 6 comprising SEQ ID NO: 1.

17. A composition comprising the recombinant AAV of claim 1.

18. A composition comprising the recombinant AAV of claim 6.

19. A method of increasing muscle mass in a subject suffering from muscular dystrophy comprising administering to the subject a therapeutically effective amount of a recombinant AAV comprising the polynucleotide sequence of claim 6.

20. A method of increasing muscle mass in a subject suffering from muscular dystrophy comprising administering to the subject a therapeutically effective amount of a recombinant AAV comprising the polynucleotide sequence of claim 17.
